(19) United States
(12) Patent Application Publication
(10) Pub. No.: US 2015/0141853 A1
(43) Pub. Date: May 21, 2015

(54) MULTI-SENSOR LESION ASSESSMENT DEVICE AND METHOD

(71) Applicant: ACIST Medical Systems, Inc., Eden Prairie, MN (US)

(72) Inventors: Edward R. Miller, III, Eden Prairie, MN (US); Sidney Donald Nystrom, Shoreview, MN (US)

(21) Appl. No.: 14/541,703

(22) Filed: Nov. 14, 2014

Related U.S. Application Data
(60) Provisional application No. 61/904,819, filed on Nov. 15, 2013.

(51) Int. Cl. A61B 5/02; A61B 5/00; A61B 5/0215

(52) U.S. Cl. CPC A61B 5/02007; A61B 5/02158; A61B 5/6851
USPC 600/486; 600/481

(57) ABSTRACT

An intravascular sensor delivery device can have a sensor that is used to measure a physiological parameter of a patient, such as blood pressure, within a vascular structure or passage. In some embodiments, the device can be used in combination with a medical guidewire carrying another sensor also configured to measure a physiological parameter of the patient, such as blood pressure. Data generated from the intravascular sensor delivery device sensor and the guidewire sensor can be used to determine a characteristic of interest for the vascular structure under investigation. For example, the data can be used to calculate a pressure distal to pressure proximal ratio across a stenotic lesion in order to assess the severity of the lesion.

### RELATED APPLICATIONS

This application claims the benefit of U.S. Provisional Application Ser. No. 61/904,819, filed Nov. 15, 2013, the contents of which are hereby incorporated by reference.

### TECHNICAL FIELD

This application relates generally to the field of medical device technology and, more particularly, to devices and methods for positioning and utilizing physiological sensors in anatomical (e.g., vascular) structures of patients, such as in blood vessels or across heart valves.

### BACKGROUND

Certain physiological measurements may be made by positioning a sensor within a patient. Such physiological measurements may include, for example, measurements of blood parameters, such as blood pressure, oxygen saturation levels, blood pH, etc. Some such measurements may have diagnostic value and/or may form the basis for therapy decisions.

A technique for evaluating the degree to which a stenotic lesion obstructs flow through a blood vessel is called the Fractional Flow Reserve measurement (FFR). To calculate the FFR for a given stenosis, two blood pressure readings are taken. One pressure reading is taken on the distal side of the stenosis (e.g., downstream from the stenosis), the other pressure reading is taken on the proximal side of the stenosis (e.g., upstream from the stenosis, towards the aorta). The FFR is defined as the ratio of maximal blood flow in a stenotic artery, taken distal to the lesion, to normal maximal flow, and is typically calculated based on a measured pressure gradient of the distal pressure to the proximal pressure. The FFR is therefore a unitless ratio of the distal and proximal pressures measured at maximum hyperemia. The pressure gradient, or pressure drop, across a stenotic lesion is an indicator of the severity of the stenosis, and the FFR is a useful tool in assessing a stenosis severity. The more restrictive the stenosis is, the greater the pressure drop, and the lower the resulting FFR. The FFR measurement may be a useful diagnostic tool. For example, clinical studies have shown that an FFR of less than about 0.75 may be a useful criterion on which to base certain therapy decisions. Pijls, DeBruyne et al., Measurement of Fractional Flow Reserve to Assess the Functional Severity of Coronary-Artery Stenoses, 334:1703-1708, New England Journal of Medicine, Jun. 27, 1996. A physician might decide, for example, to perform an interventional procedure (e.g., angioplasty or stent placement) when the FFR for a given stenotic lesion is below 0.75, and may decide to forego such treatment for lesions where the FFR is above 0.75. Thus, the FFR measurement could become a decision point for guiding treatment decisions.

### SUMMARY

In general, this disclosure is directed to devices, systems, and techniques for performing diagnostic analysis within a body of a patient, such as diagnostic analysis of a stenotic lesion in a blood vessel of the patient. Example diagnostic applications include, but are not limited to, cardiovascular procedures in coronary arteries, interventional radiology applications in peripheral arteries, and structural heart applications in heart valves.

In some examples, a guide wire that carries a sensor, such as an integrated pressure sensor in a distal portion of the guide wire, is advanced into a body lumen of a patient. The guidewire sensor may be positioned distal to a location of interest in the body lumen, such as distal to a stenosis. In addition, a sensor delivery device that carries an additional sensor may be advanced into the body lumen of the patient. The sensor delivery device may slide over the guidewire and be positioned so the sensor carried by the sensor delivery device is proximal to the location of interest in the body lumen, such as proximal to the stenosis. One or more processors communicatively coupled to the guidewire sensor and the sensor carried by the delivery device can receive a signal indicative of blood pressure measured distally to the location of interest and a signal indicative of blood pressure measured proximally to the location of interest. The one or more processors can then compare the signals to determine a characteristic of the location of interest. For example, the one or more processors may calculate a ratio of the blood pressure measured distally of the location of interest to the blood pressure measured proximally of the location of interest and determine therefrom a pressure distal (P_{d})/pressure proximal (Pₚ) ratio across the location of interest. The measurements can be taken in a non-hyperemic state, in a hyperemic state, or at maximum hyperemia. In certain embodiments, the measurements are used to determine a fractional flow reserve (FFR) of the location of interest.

Depending on the properties of the anatomical structure undergoing diagnostic analysis, the devices, systems, and techniques may be used to determine characteristics of interests for multiple locations of interest within a patient during analysis. For instance, for patients that have multiple lesions within a blood vessel, the techniques may be used to determine a characteristic of interest for each of the multiple lesions. In the case of a patient having two or more lesions separated axially along the length of a body lumen (sometimes referred to as “tandem lesions”), for example, a guidewire sensor carried by a medical guidewire may be positioned distally to the distal-most lesion under investigation. A sensor carried by a sensor delivery device can be advanced along the guidewire carrying the guidewire sensor and positioned so the sensor delivery device sensor is located between the two lesions. Further, an additional sensor can be placed in pressure communication with a location proximal to the proximal-most lesion. In one example, an additional sensor is inserted into the body lumen (e.g., using a second sensor delivery device) and positioned proximal to the proximal-most lesion. In another example, a fluid tubing is inserted into the body lumen of the patient and coupled to a hemodynamic pressure transducer located outside the body of the patient (e.g., associated with a fluid injection device). The hemodynamic pressure transducer can measure proximal to the proximal-most lesion via a column of fluid extending from the body lumen, through the fluid tubing, and back to the hemodynamic pressure sensor. In either case, one or more processors can receive a signal representative of blood pressure measured distally to a distal-most lesion, a signal representative of blood pressure measured between a distal-most lesion and proximal-most lesion, and a signal representative of blood pressure measured proximal to the proximal-most lesion. The measurements can be taken in a non-hyperemic state, a hyperemic state, or at maximum hyperemia. The one or more processors can compare the signals to determine characteristics of each of the lesions. For example, the one or more processors may determine a P_{d}/Pₚ ratio of the distal-most lesion and a P_{d}/Pₚ ratio of the proximal-most lesion. As another example, the one or more processors may calculate determine a FFR of the distal-most lesion and a FFR of the proximal-most lesion. In some embodiments, such systems, devices, and methods are useful for determining a differential pressure across any lesion in a series.

Although different devices can be used according to the disclosure, in some examples, an intravascular sensor delivery device includes a sensor delivery device having a sensor and a distal sleeve with a guidewire lumen for sliding over a medical guidewire having a sensor. The sensors (e.g., the sensor delivery device sensor and guidewire sensor) can each be adapted to measure a physiological parameter of a patient and generate a signal representative of the physiological parameter. In some embodiments, the sensor delivery device has a proximal portion coupled to the distal sleeve. The proximal portion can have a communication channel for communicating the signal from the sensor of the sensor delivery device to a location outside of the patient (such as a display monitor, or another medical device, etc.). The proximal portion of the sensor delivery device (when included) is adapted to facilitate positioning of the sensor within a vascular structure of the patient over the guidewire. Further, the guidewire can comprise a communication channel for communicating the signal from the sensor of the guidewire to a location outside of the patient (such as a display monitor, or another medical device, etc.). In some embodiments, both the signal from the sensor of the of sensor delivery device and the signal from the sensor of the guidewire are communicated to the same location, such as a processor, and calculations based on the signals are performed.

A method of assessing the severity of a stenotic lesion in a blood vessel of a patient according to some embodiments comprise deploying a guidewire with a sensor to a position such that the sensor is a position proximal of the lesion and measuring proximal (e.g., aortic) pressure. In some embodiments, the method may further include deploying an intravascular sensor delivery device having as sensor over the guidewire to a position such that the sensor is distal to the lesion, and measuring a distal pressure. In some embodiments, the method also includes calculating a ratio (or some other quantitative comparison) of the two pressure measurements.

### BRIEF DESCRIPTION OF THE DRAWINGS

FIG. 1 is a perspective view of a sensor delivery device according to an embodiment of the invention;

FIG. 2 is a conceptual perspective view of a sensor delivery device for making physiological measurements according to an embodiment of the invention;

FIG. 3 is a conceptual plot of a patient's blood pressure as a function of time;

FIG. 4A is a side view of a sensor delivery device according to an embodiment of the invention having one or more flow holes disposed along a side portion;

FIG. 4B is a cross-sectional view of a sensor delivery device according to an embodiment having one or more flow holes;

FIG. 5A is a cut-away side view of a sensor delivery device with a sensor housing according to one embodiment of the invention;

FIG. 5B is a cut-away side view of a sensor delivery device with a sensor housing according to one embodiment of the invention;

FIGS. 5C and 5D are side views of a sensor delivery device with radiopaque marker band according to certain embodiments of the invention;

FIG. 5E is a cut-away side view of a sensor delivery device with a strain relief spacer according to one embodiment of the invention;

FIGS. 6A-6G are enlarged side views of a distal transition of a sensor delivery device according to certain embodiments of the invention;

FIGS. 7A and 7B are perspective views of a sensor delivery device having a second sensor disposed on a proximal sleeve according to an embodiment of the invention;

FIG. 8 is a perspective view of a sensor delivery device having a furcation tube according to an embodiment of the invention;

FIG. 9 is a cross-sectional side view of a sensor delivery device having a dual lumen configuration according to one embodiment of the invention;

FIGS. 10A-10C are side views of a sensor delivery device having an over-the-wire configuration according to one embodiment of the invention;

FIG. 11 is a flow diagram showing a method of using a sensor delivery device according to certain embodiments of the invention;

FIG. 12 is a perspective view of a fluid injection system that may be used to interact with a sensor delivery device according to an embodiment of the invention;

FIG. 13 is a perspective view of a fluid injection system that may be used to interact with a sensor delivery device according to an embodiment of the invention;

FIG. 14 is a perspective view of a powered injection system adapted to be coupled to a physiological sensor delivery device according to certain embodiments of the invention;

FIG. 15 is an idealized view of a user interface screen containing information that may be displayed to an operator, according to certain embodiments of the invention;

FIG. 16 is a conceptual perspective view of an example system that includes sensor delivery device sensor and a guidewire sensor for making physiological measurements.

FIG. 17 is a conceptual perspective view of another example system that includes sensor delivery device sensor and a guidewire sensor for making physiological measurements.

### DETAILED DESCRIPTION

The following detailed description should be read with reference to the accompanying drawings, in which like numerals denote like elements. The drawings, which are not necessarily to scale, depict selected embodiments of the invention—other possible embodiments may become readily apparent to those of ordinary skill in the art with the benefit of these teachings. Thus, the embodiments shown in the accompanying drawings and described below are provided for illustrative purposes, and are not intended to limit the scope of the invention as defined in the claims appended hereto.

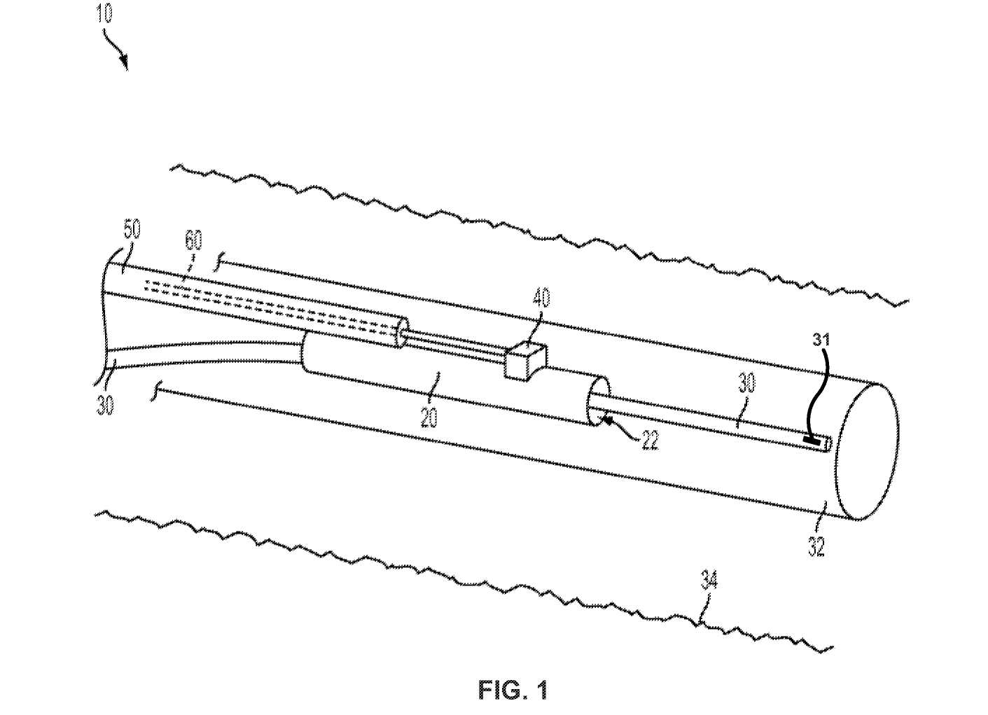

An example of a sensor delivery device according to certain embodiments is shown in FIG. 1. The sensor delivery device 10 of FIG. 1 includes a distal sleeve 20 having a guidewire lumen 22 for slidably receiving a medical guidewire 30. The sensor delivery device includes a sensor 40. In the embodiment shown, the sensor 40 is coupled to the distal sleeve 20. In other embodiments, sensor 40 can be coupled to other portions of the sensor delivery device. Sensor 40 can be capable of sensing and/or measuring a physiological parameter of a patient and generating a signal representative of the physiological parameter. Thus, the distal sleeve 20 and the sensor 40 may be positioned within a patient (e.g., within an anatomical structure of a patient, such as within a vein, artery, or other blood vessel, or across a heart valve, for example) by causing the distal sleeve 20 to slide over the medical guidewire 30 to the desired position.

The sensor delivery device 10 of FIG. 1 also includes a proximal portion 50, which is coupled to the distal sleeve 20. The proximal portion 50 includes a communication channel 60 for communicating the signal from the sensor 40 to a location outside of the patient (e.g., to a processor, display, computer, monitor, or to another medical device). Communication channel 60 may comprise a fiber optic communication channel in certain preferred embodiments, such as where the sensor 40 is a fiber optic pressure sensor. Alternately, communication channel 60 may comprise an electrically conductive medium, such as one or more electrical conducting wires. Of course, many other forms of communication media may be suitable for transmitting the signal generated by sensor 40 to a location outside of the patient. In some embodiments of the invention, the communication channel 60 may comprise any of a variety of fluid and/or non-fluid communication media, such as a wireless communication link, or an infrared capability, or acoustic communications such as ultrasound, as possible examples.

The proximal portion 50 is also adapted to assist an operator (e.g., a physician or other medical staff) in positioning the distal sleeve 20 and the sensor 40 within an anatomical (e.g., vascular) structure of the patient. This is typically accomplished by an operator first inserting a medical guidewire 30 into a patient's vasculature and advancing it past an area of interest. The sensor delivery device 10 is then deployed by “threading” the distal sleeve 20 onto the guidewire 30 such that the lumen 22 slides over the guidewire 30, and advancing the distal sleeve 20 (and the associated sensor 40) by moving (e.g., pushing and/or pulling) the proximal portion 50 until sensor 40 is in the desired location.

The device 10 and the guidewire 30 are typically manipulated inside a guiding catheter 32, which has been placed in the anatomical (e.g., vascular) structure of interest. In certain embodiments of the invention, the guidewire lumen 22 may be sized to slide over medical guidewires having a specific size. A device according to embodiments of the invention may therefore be made available in a range of sizes corresponding to different medical guidewire sizes.

In general, guidewire 30 provides a surface or rail over which device 10 is advanced to position sensor 40 at a desired location within an anatomical structure of a patient. Guidewire 30 may carry an additional sensor 31 that is independently positionable from sensor 40. Sensor 31 may be integrated with guidewire 30 such that the sensor is not separable from the guidewire during standard use of the guidewire. To facilitate communication from sensor 31 to a location outside the body of a patient, guidewire 30 may include a communication channel running along the length of the guidewire (not illustrated in FIG. 1). The communication channel which, in different examples can be a channel that communicates electrical signals or optical signals, provide signal communication between sensor 31 and a device located outside of the patient. In one example, guidewire 30 is implemented as a pressure sensing guidewire and sensor 31 is configured to measure blood pressure of a patient.

When used, sensor 31 can be positioned at any suitable location along the length of guidewire 30. Typically, sensor 31 is positioned in a distal portion of guidewire 30 that is closer to a distal terminal end of the guidewire than a proximal terminal end of the guidewire, e.g., when the guidewire is inserted so the distal end is advanced in a leading direction into the body of the patient. For example, sensor 31 may be positioned in a distal end of guidewire 30 that extends beyond sensor delivery device 10, as illustrated in FIG. 1.

In the example shown in FIG. 1, the device 10 is being deployed using guiding catheter 32, which has been placed within a vascular structure of interest (in this example, blood vessel 34, which could be, for example, a coronary artery of the patient). In certain embodiments of the invention, the size or “footprint” (e.g., the width and/or the cross-sectional area) of device 10 may allow it to fit within certain standard sized guiding catheters. For example, in certain diagnostic applications, it would be desirable to have device 10 deployed within a certain sized guiding catheter (e.g., smaller than about 4 or 5 French (FR)).

In certain embodiments of the invention, the distal sleeve 20 of the device may be substantially concentric with the guidewire 30. The coupling of the proximal portion 50 to the distal sleeve 20 allows the guidewire 30 to separate from the rest of device 10 (e.g., in what is sometimes referred to as a “monorail” catheter configuration); this would typically occur inside the guiding catheter 32. The guidewire 30 and device 10 would both exit the patient at the proximal end of the guiding catheter 32 as separate devices. Having the device 10 and guidewire 30 separate allows the physician to independently control device 10 and guidewire 30, as necessary.

One diagnostic application in which various embodiments of the invention may be well-suited is the measurement of P_{d}/Pₚ and/or Fractional Flow Reserve (FFR). As noted above, the P_{d}/Pₚ ratio quantifies the degree to which a stenotic lesion, for example, obstructs flow through a blood vessel. To calculate the P_{d}/Pₚ ratio for a given stenosis, two blood pressure measurements are needed: one pressure reading is taken on the distal side of the stenosis (downstream side), the other pressure reading is taken on the proximal side of the stenosis (upstream side). The P_{d}/Pₚ ratio is therefore a unitless ratio of the distal pressure to the proximal pressure. The pressure gradient across a stenotic lesion is an indicator of the severity of the stenosis. The more restrictive the stenosis is, the more the pressure drop, and the lower the P_{d}/Pₚ ratio.

To add clarity and context to the disclosure, several embodiments of the invention will now be described below in the context of making P_{d}/Pₚ ratio measurements. However, it should be realized that there are other applications in which physiological parameter measurements could be facilitated with the devices and/or methods described herein.

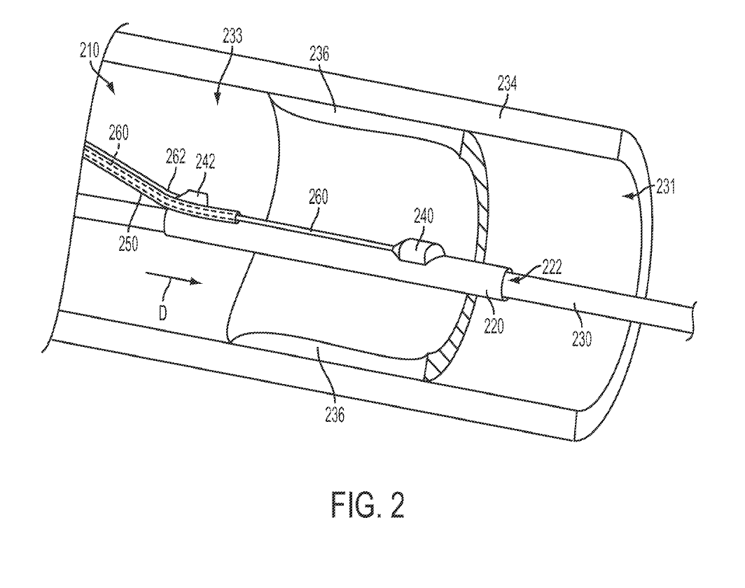

FIG. 2 is a perspective view of a sensor delivery device for measuring a physiological parameter in a patient. The embodiment shown in FIG. 2 might, for example, be deployed to make a P_{d}/Pₚ measurement in a blood vessel of a patient. FIG. 2 shows a sensor delivery device 210 being deployed in a blood vessel of a patient (e.g., coronary artery 234) across a stenosis (e.g., stenotic lesion 236). To make an P_{d}/Pₚ ratio measurement, for example, first sensor 240 may be positioned to measure distal (downstream) blood pressure, P_{d}, at a location 231 downstream of a location of interest (e.g., stenotic lesion 236). And/or first sensor 240 may be positioned to measure proximal (upstream) blood pressure, Pₚ, at a location 233 upstream of a location of interest (e.g., stenotic lesion 236). The P_{d}/Pₚ ratio is simply calculated as the ratio of distal pressure to proximal pressure, in this embodiment using at least one value obtained from the sensor of the sensor delivery device. As discussed further below, the other value can be obtained from a pressure transducer associated with an injection system and/or a sensor included in the guidewire used to position the sensory delivery device. The use of the terms “downstream” and “upstream” are with respect to the normal direction of blood flow, “D,” as shown in FIG. 2.

In some embodiments, first sensor 240 coupled to sensor delivery device 210 (which may or may not be the only sensor carried by the sensor delivery device) can be positioned proximal to a location of interest, such as at a location 233 proximal to stenotic lesion 236. Further, a sensor carried by guidewire 230 can be positioned distal to the location of interest, such as at location 231 distal to stenotic lesion 236. First sensor 240 carried by sensor delivery device 210 can measure a proximal blood pressure, Pₚ, and a sensor carried by the guidewire can measure a distal pressure, P_{d}. One or more processors communicatively coupled to the sensor delivery device 210 and guidewire 230 can receive signals representative of the proximal and distal blood pressures and calculate P_{d}/Pₚ ratios therefrom.

The sensors can be adapted to measure a physiological parameter of a patient, such as a blood parameter (e.g., blood pressure, temperature, pH, blood oxygen saturation levels, etc.), and generate a signal representative of the physiological parameter. In certain preferred embodiments of the invention, the sensors include a fiber optic pressure sensor adapted to measure blood pressure. An example of a fiber optic pressure sensor is a Fabry-Perot fiber optic pressure sensor, which is a commercially available sensor. Examples of Fabry-Perot fiber optic sensors are the “OPP-M” MEMS-based fiber optic pressure sensor (400 micron size) manufactured by Opsens (Quebec, Canada), and the “FOP-MIV” sensor (515 micron size) manufactured by Fiso Technologies, Inc. (Quebec, Canada). In certain alternate embodiments, sensors may also include a piezo-resistive pressure sensor (e.g., a MEMS piezo-resistive pressure sensor), and in other embodiments, sensors may include a capacitive pressure sensor (e.g., a MEMS capacitive pressure sensor). A pressure sensing range from about −50 mm Hg to about +300 mm Hg (relative to atmospheric pressure) is desired for making most physiological measurements with a sensor, for example.

In embodiments of the invention using the Fabry-Perot fiber optic pressure sensor, such a sensor works by having a reflective diaphragm that varies a cavity length measurement according to the pressure against the diaphragm. Coherent light from a light source travels down the fiber and crosses a small cavity at the sensor end. The reflective diaphragm reflects a portion of the light signal back into the fiber. The reflected light travels back through the fiber to a detector at the light source end of the fiber. The two light waves, the source light and reflected light travel in opposite directions and interfere with each other. The amount of interference will vary depending on the cavity length. The cavity length will change as the diaphragm deflects under pressure. The amount of interference is registered by a fringe pattern detector.

In FIG. 2, first sensor 240 is coupled to distal sleeve 220. In the embodiment first sensor 240 is coupled to an outer surface of distal sleeve 220. FIG. 2 also shows proximal portion 250 coupled to the distal sleeve 220. The proximal portion 250 includes a communication channel 260 for communicating the physiological signal from the sensor 240 to a location outside of the patient (e.g., to a processor, display, computer, monitor, or to another medical device). The proximal portion 250 may preferably be formed of a material of sufficient stiffness in order to assist an operator (e.g., a physician or other medical staff) in positioning the distal sleeve 220 and the sensor 240 within an anatomical (e.g., vascular) structure of the patient.

One suitable material for the proximal portion 250 may be a stainless steel hypotube, for example. Depending on the application, the proximal portion 250 (sometimes also referred to as the “delivery tube”) should typically be stiffer and more rigid than the distal sleeve 220 in order to provide a reasonable amount of control to push, pull and otherwise maneuver the device to a physiological location of interest within the patient. In interventional cardiology procedures, for example, at least a portion of the proximal portion 250 will be maneuvered within a guiding catheter positioned within the aortic artery. The proximal portion 250 in such an application should therefore be flexible enough to accommodate the arch of the aorta, while being rigid enough to push and pull the device. Accordingly, suitable materials for proximal portion 250 may also include (in addition to the aforementioned stainless steel hypotube) materials such as nitinol, nylon, and plastic, for example, or composites of multiple materials.

The communication channel 260 may be disposed along an outer surface of proximal portion 250, or may be formed within the proximal portion 250, as shown in FIG. 2. For example, communication channel 260 may comprise a communication lumen that extends longitudinally through proximal portion 250 in some embodiments. Communication channel 260 may comprise a fiber optic communication channel in certain embodiments, such as where the sensor 240 is a fiber optic pressure sensor. Alternately, communication channel 260 may comprise an electrically conductive medium, such as electrical conducting wires, or other communication media suitable for transmitting the signal generated by sensor 240. In preferred embodiments of the invention, the communication channel 260 comprises a non-fluid communication medium. In the embodiment shown in FIG. 2, communication channel 260 (e.g., a fiber optic cable) extends distally beyond proximal portion 250 and is coupled to sensor 240. The communication channel 260 in such an embodiment is at least partially housed within a communication lumen of the proximal portion 250 (e.g., a stainless steel hypotube).

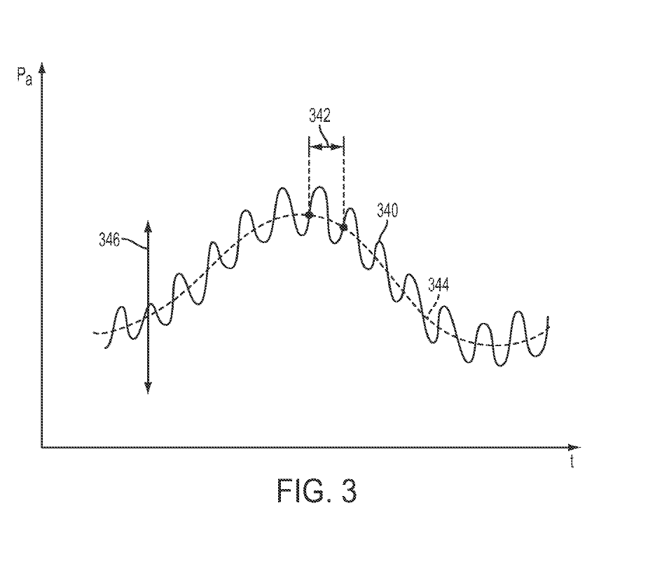

FIG. 2 also shows an optional embodiment of the invention in which a second sensor 242 may be coupled to the device 210. For example, a second sensor 242 may be coupled to proximal portion 250 such that the first and second sensor 240, 242 are spaced apart sufficiently (e.g., a fixed distance apart) to span a stenotic lesion. Second sensor 242 may have a communication channel 262, which could be housed within proximal portion 250, or could be disposed along an outside surface of proximal portion 250, as shown in FIG. 2, for example. Further, the ability to measure P_{d} and Pₚ substantially simultaneously may improve accuracy and/or reduce the effects of certain types of errors illustrated and described below with reference to FIG. 3. In other examples, device 210 does not have second sensor 242 or only has second sensor 242 in lieu of first sensor 240, in which case the second sensor can be referred to as a first sensor 242.

It should be noted that certain embodiments could have more than 2 sensors, and that the spacing between adjacent sensors in such embodiments may be varied to provide a variable spacing capability. In certain alternate embodiments of the invention, one or more sensors could be disposed on the proximal portion 250 with no sensors disposed on the distal sleeve 220, for example. In some alternate embodiments, it may be desirable to have a plurality of sensors (two, or three, or four, or more sensors) spaced at known, fixed distances, disposed along the proximal portion 250. This could, for example, provide the ability to measure P_{d} and Pₚ substantially simultaneously, regardless of lesion length, by selecting an appropriate pair of sensors (from among the plurality of sensors) placed across the lesion from which to obtain the P_{d} and Pₚ signals. Further, the sensors could have some form of radiopaque markings incorporated thereon (e.g., marker bands), which could provide a visual estimate of lesion size in conjunction with the measurement of physiological parameters (e.g., P_{d} and Pₚ).

FIG. 3 graphically illustrates several possible sources of error in measuring blood pressure, particularly as they may affect the calculation of P_{d}/Pₚ and/or FFR, for example. FIG. 3 is a conceptual plot of blood pressure, 340, as a function of time for a given patient, P(t). One potential error in calculating P_{d}/Pₚ and/or FFR is due to the fluctuations in blood pressure due to the systolic and diastolic phases of the cardiac cycle 342. Unless P_{d} and Pₚ are measured at substantially the same phase of the cardiac cycle 342, there may be some amount of error introduced. Similarly, a more slowly varying source of error can also be introduced by the effect of the respiratory cycle (e.g., inspiration and expiration) on blood pressure, as illustrated at 344 in FIG. 3. A third source of error could be introduced by changes in the patient's posture, which could either raise or lower the overall pressure profile as indicated at 346 in FIG. 3. Embodiments of the invention which have the ability to measure P_{d} and Pₚ substantially simultaneously may be able to minimize or eliminate the effects of such “timing errors” on the P_{d}/Pₚ and FFR calculations. Another method of addressing the effects of such “timing errors” will be discussed below in the context of using a contrast injection system in conjunction with a sensor delivery device, according to some embodiments of the invention.

Referring again to FIG. 2, distal sleeve 220 may be substantially tubular, as shown, or may have any shape that allows distal sleeve 220 to slide over a medical guidewire 230 in an anatomical (e.g., vascular) structure of interest. In the context of measuring P_{d}/Pₚ in a coronary artery, for example, it may be desirable that distal sleeve 220 be substantially cylindrical in cross-section to minimize the total cross-sectional area of the device. Distal sleeve 220 may be preferably formed of a flexible material in some embodiments to facilitate positioning and placement of the distal sleeve 220 (and sensor 240) over a guidewire 230 through narrow vascular structures such as coronary arteries. In certain preferred embodiments, the distal sleeve 220 comprises a flexible polyimide tube sized for placement in anatomical (e.g., vascular) structures of interest, such as in coronary arteries or peripheral arteries. In some embodiments, the distal sleeve 220 may comprise a flexible microcoil tube. In some embodiments, flexibility may be achieved and/or enhanced by applying a series of cuts along the surface of the tube. For example, a plurality of cuts or notches along a length of the outer surface of distal sleeve 220 may be applied (e.g., by laser cutting techniques known to those of ordinary skill in this field). Such cuts or notches may be substantially circumferentially directed, and may extend at least partially around the circumference of the distal sleeve. Successive cuts may be angularly offset from each other to provide flexibility in all directions according to some embodiments.

The length of distal sleeve 220 may vary. In embodiments to be used in coronary arteries, for example, distal sleeve 220 may be up to about 15 inches long, and in some preferred embodiments may be 11 inches long (e.g., to facilitate use deep within certain coronary arteries). In some embodiments, the distal sleeve 220 may also include a thin covering to provide additional structural support and/or improve handling characteristics of the device. Such a covering may comprise, for example, polyester (PET) shrink tubing that substantially covers the distal sleeve.

Distal sleeve 220 has a guidewire lumen 222 that is sized to slidably receive a guidewire 230. Guidewire 230 may have an outer diameter between about 0.010 inches and 0.050 inches, although other sizes are also possible. For making a P_{d} or Pₚ measurement in a coronary artery 234, for example, the guidewire 230 may have an outer diameter of 0.014 inches, and guidewire lumen 222 would therefore need to have an inner diameter slightly larger than this to facilitate slidable movement of the distal sleeve 220 over the guidewire 230. In examples in which guidewire 230 has an integrated sensor in a distal portion of the guidewire, the guidewire may have an enlarged outer diameter in the region of the sensor. To slide distal sleeve 220 over guidewire 230 in such an example, the guidewire lumen 222 may be sized at least as large as the enlarged cross-sectional area of the guidewire in the region of the sensor.

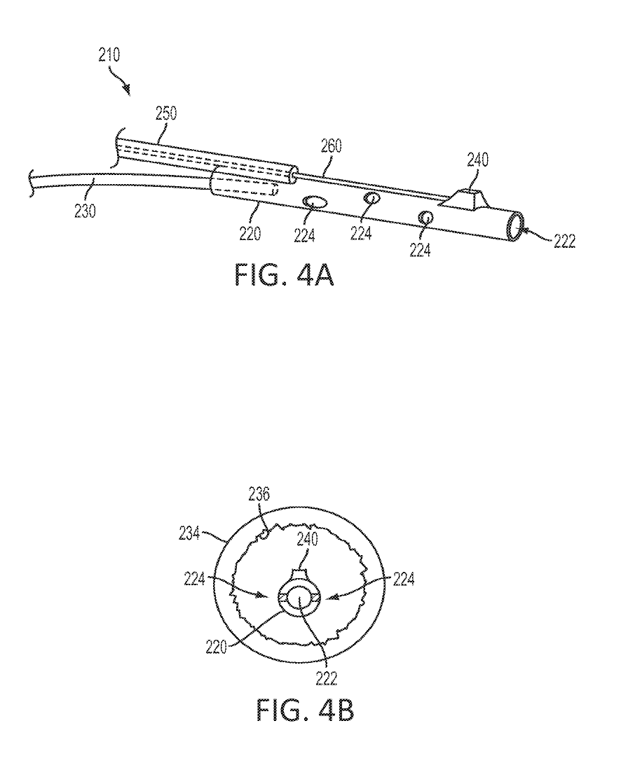

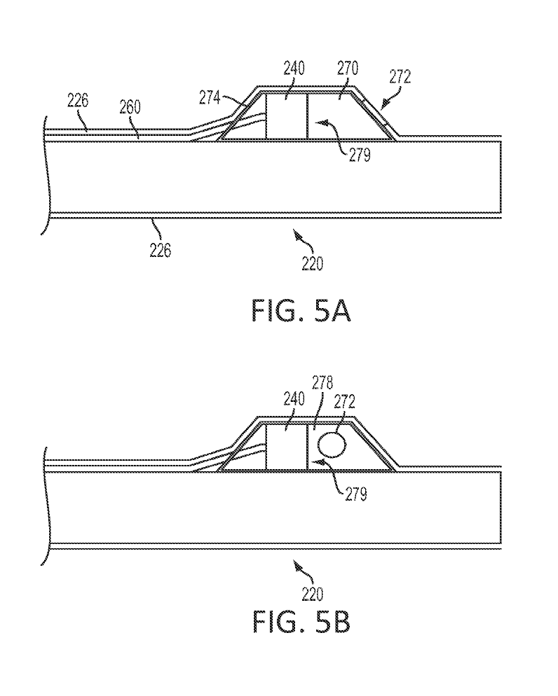

FIG. 4A shows an embodiment of the invention in which one or more flow holes 224 are disposed along a side portion of the distal sleeve 220 (e.g., along the length of distal sleeve 220). Flow holes 224 could allow blood to flow into the guidewire lumen 222 if an operator were to pull back (e.g., withdraw) the guidewire 230 as shown in FIG. 4A. Such an embodiment may provide an improvement in accuracy in measuring the pressure drop across a stenosis, since the pressure drop attributable to the device itself would be lessened by decreasing the effective cross-sectional area of the device.

FIG. 4B is a cross-sectional view of an embodiment, illustrating the potential reduction in cross-sectional area that could be obtained by employing flow holes 224 in a side portion of distal sleeve 220. For example, by allowing blood to flow through flow holes 224 into guidewire lumen 222, the effective cross-sectional area of the device 210 is reduced by the area of guidewire lumen 222, and any error in blood pressure measurements caused by the flow obstruction of the device 210 itself would be accordingly reduced.

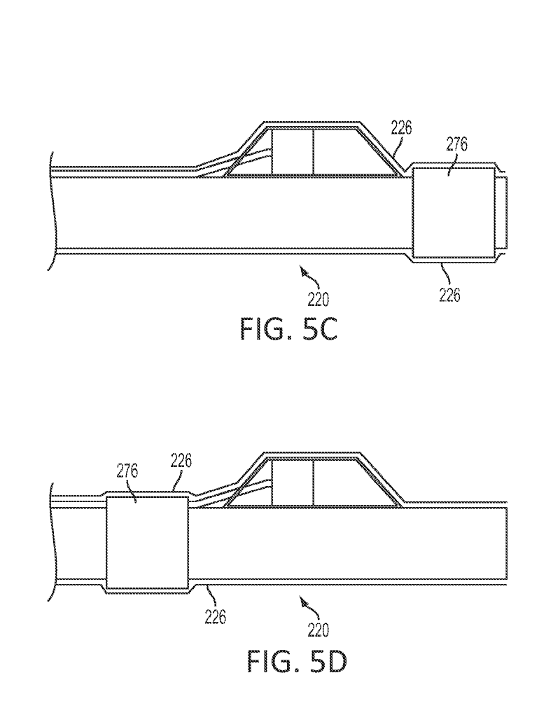

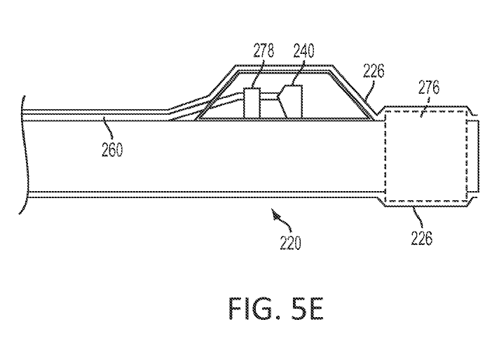

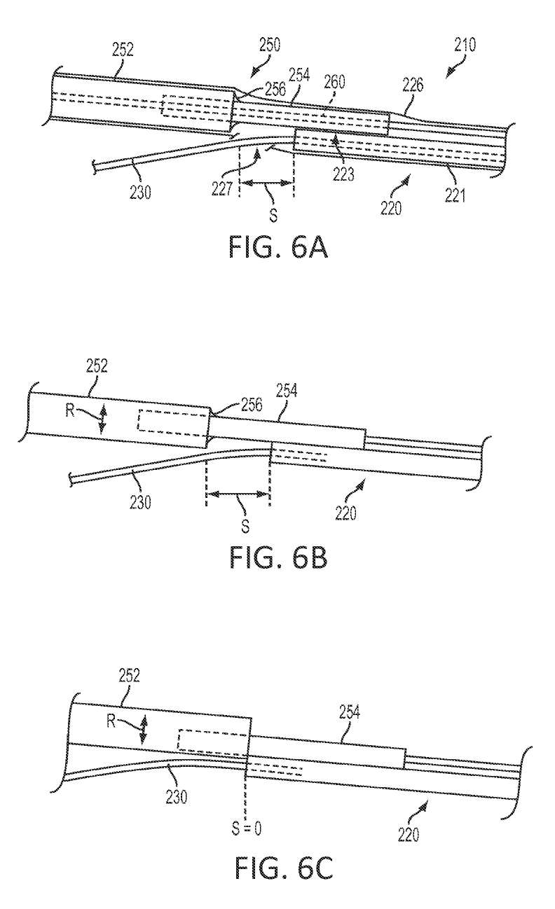

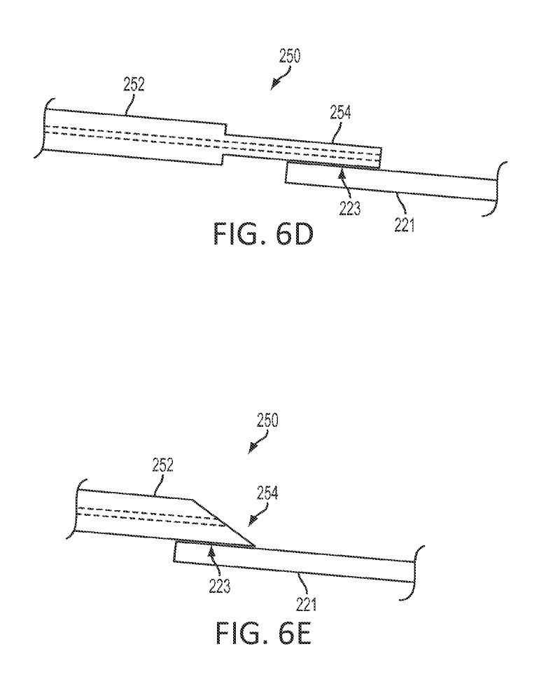

FIG. 5A is a cut-away side view of a portion of the device 210 according to certain embodiments of the invention. FIG. 5A shows the distal sleeve 220 and first sensor 240 of an embodiment in which sensor 240 is provided with a certain degree of protection by being at least partially covered by a sensor housing 270 disposed on distal sleeve 220. Sensor housing 270 may be substantially tubular, or may be semi-circular, or may be any other shape that provides suitable protection for sensor 240. Sensor housing 270 may be constructed of tubing such as polyimide, which is capable of being formed with a relatively thin wall thickness.

The sensor housing 270 may be constructed in several different ways, as described with reference to FIGS. 5A through 5E. Fiber optic sensors, for example, may be somewhat fragile, and should typically be provided with some form of mechanical protection from stress and/or strain relief. The sensing head of sensor 240 is generally attached to the communication channel 260 (e.g., a fiber optic cable) with an adhesive. The sensing head can be prone to being pulled away from (e.g., disconnected from) the fiber optic without much force because the bonding area is typically very small. FIGS. 5A through 5E illustrate several techniques that utilize a protective sensor housing 270 surrounding the sensor 240 to minimize or eliminate the effects of such stresses on the sensor 240.

One material which may be used to construct the sensor housing 270 is a heavy metal that is x-ray visible, such as platinum. A sensor housing 270 formed of platinum may provide an x-ray marker band to facilitate the placement and positioning of the sensor 240. A platinum sensor housing 270 may be formed so it is generally thin, for example, approximately 0.001 inches in thickness. Such a thin-walled platinum sensor housing 270 may provide suitable protection to the sensor 240 from stresses that might otherwise cause it to detach from the communication channel 260.

In some embodiments, sensor housing 270 may be shaped to facilitate movement and placement of the device in the anatomical (e.g., vascular) structure of the patient. For example, as shown in FIG. 5A, the forward and rearward portions 274 of sensor housing 270 may be formed at an angle (e.g., cut at an angle) to present a smoother, tapered structure that is easier to navigate through anatomical (e.g., vascular) structures and passages in a patient (e.g., it allows the device 210 to slide through vascular passages such as arterial walls without catching or snagging).

In some embodiments, sensor housing 270 may be formed as part of the process of forming distal sleeve 220. For example, a substantially cylindrical mandrel may be used to form a distal sleeve 220 made of a thermoset polymer (e.g., polyimide) by employing a dipping process. A slight modification of this manufacturing process could employ a “housing forming element” located alongside the mandrel at the distal end of the mandrel. A single dipping process could thereby form sensor housing 270 as an integral part of distal sleeve 220.

In some embodiments, an optional covering 226 may be applied over the sensor housing 270 and distal sleeve 220. Such a covering 226 may facilitate movement and positioning of the device 210 within an anatomical (e.g., vascular) structure of a patient. The covering 226 may also provide additional structural stability to the sensor 240, housing 270, and distal sleeve 220 arrangement. An example of a class of materials that may be suitable for forming covering 226 are thermoplastics. Such materials may sometimes be referred to as thin-walled heat-shrink tubing, and include materials such as polyolefin, fluoropolymers (PTFE), polyvinyl chloride (PVC), and polyester, specifically polyethylene terephthalate (PET). For simplicity, the term “PET tubing” will be used herein in reference to embodiments that incorporate such thin covering materials. The use of PET tubing could be employed, for example, in embodiments with or without a housing 270.

PET tubing is a heat shrink tube made from polyester that exhibits excellent tensile strength characteristics, while having a wall thickness as little as 0.0002 inches. PET tubing may be used in some embodiments of the invention to encapsulate the distal sleeve 220. This may include, for example, encapsulating the sensor housing 270 and/or a portion of the communication channel 260 (e.g., the fiber optic cable), to the extent the communication channel 260 extends from the proximal portion 250. In some embodiments, the PET tubing may also extend to cover part of the proximal portion 250, for example, where it is coupled to the distal sleeve 220. In some embodiments, PET tubing may be used to hold a fiber optic communication channel 260 in place around the distal sleeve 220. After the PET tubing has been heat shrunk, one or more openings may be cut in the PET tubing, for example, to allow an exit port for the guidewire 230.

FIG. 5A shows a fluid opening 272 formed in one of the portions 274 (e.g., the forward portion in this example) of the sensor housing 270. Fluid opening 272 allows fluid (e.g., blood) to enter the sensor housing 270 and come into fluid contact with sensor 240. In embodiments that incorporate a covering 226 (such as PET tubing), fluid opening 272 may be formed in the covering 226.

FIG. 5B shows an embodiment of the invention where the fluid opening 272 is formed in a side portion of the housing 270. This arrangement may provide a reduced likelihood of “clogging” within sensor housing 270, and/or a reduced likelihood of catching or snagging on any obstructions or bends encountered while positioning device 210. For example, plaque or calcium from arterial walls may enter the housing 270 as the device is moved through an artery; having the fluid opening 272 in a side portion of housing 270 may reduce this effect. In some embodiments, allowing the PET tubing covering 226 to remain intact at the distal end of the housing 270 may prevent foreign material from entering the housing 270 and possibly damaging the sensor 240, or affecting the accuracy of pressure measurements. After the PET tubing covering 226 has been heat shrunk over the device 210, holes can be punched through the covering 226 as needed to form fluid openings 272 to allow fluid access (e.g., blood flow) inside the sensor housing 270.

In some embodiments of the invention, the inside portion of the sensor housing 270 may be filled with a gel 278, such as a silicone dielectric gel. Silicone dielectric gels are often used with solid state sensors to protect the sensor from the effects of exposure to a fluid medium, for example. If the sensor housing 270 is filled with a gel 278 in front of the sensor diaphragm 279, then foreign material would be less likely to penetrate inside the housing 270. The gel 278 may also offer added structural stability to the sensor 240, and/or may enhance the pressure-sensing characteristics of the sensor 240. A gel 278 may be used in any of the embodiments of sensor housing 270 illustrated in FIGS. 5A to 5D and their equivalents.

In FIGS. 5C and 5D, embodiments of the invention are shown which include an optional marker band. If the sensor housing 270 is made from polyimide tubing, for example, the device 210 may not show up as well under x-ray. An optional marker band 276 could be placed near the end of the distal sleeve 220. Marker band 276 may provide a visible indication of the location of the sensor 240 when viewed under x-ray. As shown in FIG. 5C, the marker band 276 on the end of the distal sleeve 220 may provide some structural reinforcement to the end of the distal sleeve 220. In the alternative embodiment shown in FIG. 5D, a marker band 276 on the distal sleeve 220 located proximal of the sensor housing 270 may reduce the likelihood of the marker band 276 becoming dislodged from the device 210. In some embodiments, it may be desirable to include a number of such marker bands spaced at known distances (e.g., every 10 mm along distal sleeve 220, for example), such that the marker bands could be used to provide visual estimates of length or distance (e.g., to measure lesion length).

FIG. 5E shows an embodiment where a spacer 278 is used to provide strain relief at the connection between the sensor 240 and the communication channel 260. This strain relief may be made of any suitable material, such as polyetheretherketone (PEEK), for example. In some embodiments, spacer 278 may also be formed so as to serve as a marker band 276, substantially as described above. Spacer 278 could be employed in embodiments with a sensor housing 270, or in embodiments without a sensor housing.

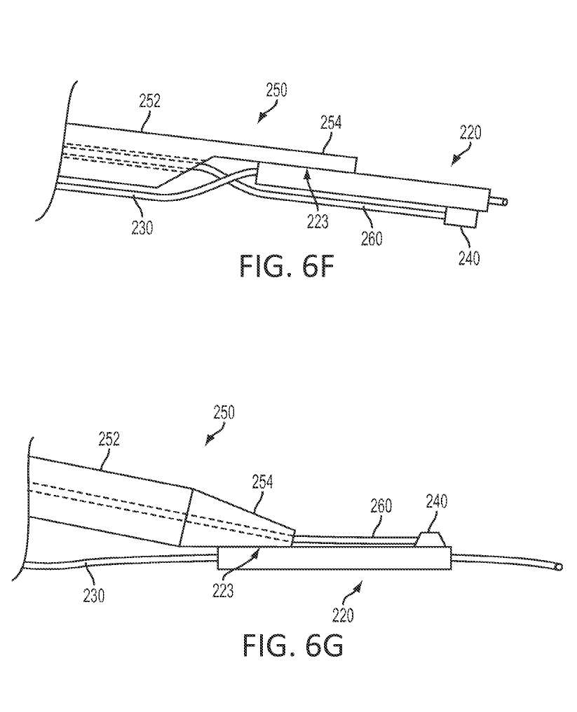

FIG. 6A shows an enlarged side view of a portion of the device 210 according to one embodiment of the invention. The delivery tube (proximal portion 250) and distal sleeve 220 are preferably coupled together using a flexible bond method (medical adhesive) to maintain flexibility of the device 210. In some preferred embodiments, for example, the proximal portion 250 will be bonded to an outer surface 221 of the distal sleeve 220 in a bonding area 223. Bonding area 223 is preferably disposed on distal sleeve 220 sufficiently proximal of the sensor 240 so that bonding area 223 is not within the vascular structure or passage of interest (e.g., it is not within the arterial vessel near a stenosis), but would still be inside the guiding catheter 232. The joining or bonding area 223 preferably maintains a degree of flexibility in order to accommodate bends such as that in the aortic arch. As previously noted, it may be desirable to minimize the width of the device 210 so that it can be passed through a relatively small guiding catheter 232, for example. This goal may be achieved, at least in part, by causing the bonding area 223 to be as narrow as possible. In some embodiments, it is desirable to use the sensor delivery device 210 inside a diagnostic guiding catheter 232, which are generally 4 Fr.

In some embodiments, the use of a distal transition 254 to couple the proximal portion 250 to the distal sleeve 220 may obtain a significant reduction in the width of the device 210. In certain preferred embodiments of the invention, the device 210 will be able to pass through a 4 Fr guiding catheter 232. The embodiment of FIG. 6A has a proximal portion 250 that comprises a main section 252 and a distal transition 254. Distal transition 254 extends distally from main section 252 and is coupled to an outer surface 221 of distal sleeve 220 at bonding area 223. As shown in FIG. 6A, the use of a distal transition 254 to couple the proximal portion 250 to the distal sleeve 220 may cause a reduction in the width of the device 210 as compared to a device 210 without the distal transition 254. This may be accomplished, for example, in embodiments where the distal transition 254 is smaller in cross-sectional area than main section 252. (Of course, the distal transition 254 is optional and may not be required in all embodiments of the invention; the embodiments shown in FIGS. 1, 2, and 4, for example, do not include a distal transition. Such embodiments may result in a simpler manufacturing process, for example.)

In the embodiment shown in FIG. 6A, distal transition 254 may be substantially coaxial and/or concentric with main section 252, and is smaller in diameter than main section 252. In some embodiments, distal transition 254 may be formed by inserting a hypotube inside the end of the proximal portion 250, the hypotube being of somewhat smaller diameter than the proximal portion 250. The hypotube distal transition 254 and the proximal portion may then be soldered together, as shown at 256. The distal sleeve 220, which may comprise a thin walled tube formed of a material such as polyimide, may then be bonded to the smaller diameter distal transition 254. Alternately, the distal sleeve 220 could be formed from a flat wire wound microcoil with PET tubing heat shrunk over the microcoil. An embodiment using a stainless steel microcoil for the distal sleeve 220 might provide a lower coefficient of friction (than polyimide, for example) to reduce the sliding friction. However, such a microcoil embodiment would probably benefit from the use of a PET tubing covering 226 to provide reinforcement and/or a smooth surface. PET tubing may be used to form covering 226, as shown in FIG. 6A, and substantially as described above. Once the PET tubing covering 226 has been heat shrunk in the area of distal transition 254, for example, covering 226 may have one or more openings 227 formed in the PET tubing, for example, to create an exit port 227 for the guidewire 230, as shown. Note that, although only shown in FIG. 6A, the embodiments shown in FIGS. 6A, 6B, and 6C may all include an optional covering 226 (e.g., PET tubing), according to certain embodiments of the invention.

FIG. 6B shows an embodiment of the invention in which the longitudinal axis of distal transition 254 is offset radially some distance “R” from the longitudinal axis of main section 252 to provide a further potential reduction in the width of device 210, for example, to minimize the footprint of device 210 and allow the use of a relatively small guiding catheter. FIG. 6C shows an embodiment where the radial offset “R” is in an opposite direction from the offset “R” shown in FIG. 6B. This arrangement may provide more clearance for guidewire 230 as it exits distal sleeve 220 in the area near distal transition 254.

FIGS. 6A and 6B also illustrate techniques that may be employed to form the distal transition 254. For example, the distal transition 254 may be formed by welding or soldering a tubular member to the main section 252 as shown at 256. As shown, the tubular member 254 may extend into the end of main section 252, and may include a communication channel 260 (e.g., an extension of communication channel 260 within main section 252). Alternately, the distal transition 254 may be formed by “swaging” a distal end of the main section 252, as shown at 256. “Swaging,” as that term is used herein, encompasses a number of manufacturing processes that reduce the diameter of a workpiece, for example, by forcing the workpiece (or a portion thereof) through a confining die, or by hammering a round workpiece into a smaller diameter workpiece (e.g., rotary swaging or radial forging, for example).

Other methods of forming the distal transition 254 may include grinding (e.g., to reduce the outer diameter of a single piece from that of main section 252 to that of distal transition 254), or the use of adhesives or glue (e.g., epoxy, ultraviolet adhesives, cyanoacrylates, etc.), or thermoforming, and/or other techniques known to those of ordinary skill in this area. FIGS. 6D and 6E show exemplary embodiments that may be formed by grinding or other comparable techniques, for example. Further, distal transition 254 need not extend into the main section 252 and could instead be held in an abutting relationship to main section 252 using certain of the aforementioned techniques.

FIGS. 6A and 6B happen to show embodiments of the invention in which a distal transition 254 is employed to “setback” the main section 252 from the distal sleeve 220 a distance “S” as shown. This may, for example, be advantageous in creating additional “clearance” for the guidewire 230 as it exits the distal sleeve 220. However, the setback is not a requirement, and embodiments of the invention may be employed with a zero setback, as shown in FIG. 6C (e.g., S=0).

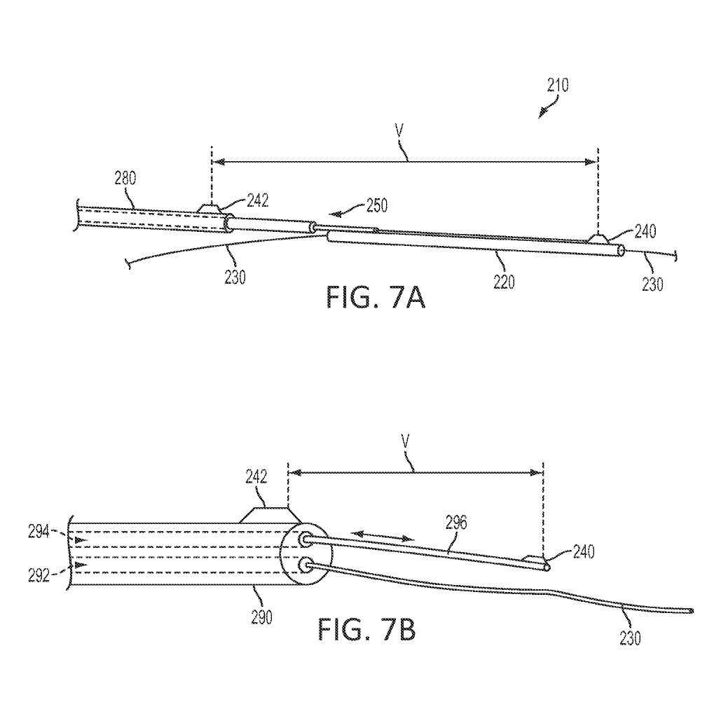

FIG. 7A shows one possible embodiment of the invention in which a second sensor 242 is coupled to a proximal sleeve 280, which thereby allows the first and second sensors 240, 242 to be spaced apart a variable distance, “V,” as shown. Proximal sleeve 280 in such an embodiment is adapted to be moved longitudinally (e.g., advanced and/or refracted) by an operator by sliding over proximal portion 250 to achieve the desired spacing, “V,” as shown.

FIG. 7B shows an alternate embodiment in which a multilumen shaft 290 (e.g., formed of a polymer) includes a guidewire lumen 292, a sensor lumen 294 for an extendible/retractable first sensor 240 disposed on a distal end of an extendible/retractable sensor shaft 296, the sensor shaft 296 being slidably received within sensor lumen 294, and a second sensor 242 coupled to an outer portion of the multilumen shaft 290. The first and second sensors 240, 242 may be spaced a variable distance apart (e.g., across a stenotic lesion of other anatomical locations of interest in a patient) by slidably moving the sensor shaft 296 with respect to the multilumen shaft 290 (e.g., by moving sensor shaft 296 within sensor lumen 294).

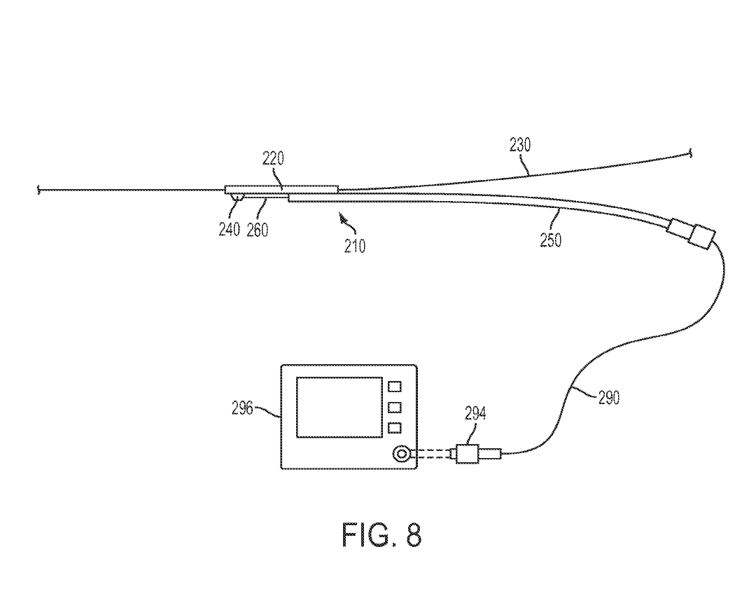

FIG. 8 shows a device 210 according to an embodiment of the invention in which a proximal end of proximal portion 250 interconnects with a fiber optic furcation tube 290 (e.g., in embodiments of the invention employing a fiber optic sensor). A fiber optic furcation tube 290 provides an extension of the fiber optic communication channel 260 (from the sensor 240 through the proximal portion 250), to an optional connector 294, such as an “SC” fiber optic connector. (An SC connector is a fiber optic connector with a push-pull latching mechanism which provides quick insertion and removal while also ensuring a positive connection. It also follows certain industry standards, allowing interconnection with a variety of fiber optic devices which follow the same standards.) Furcation tube 290 may, for example, be provided with SC connector 294 to allow the device 210 to send a signal from sensor 240, for example, to other devices, monitors, fluid injection devices, display and control units, etc. Furcation tube 290 may comprise a Kevlar fiber reinforced tube (e.g., for strength) according to some embodiments. In some alternate embodiments, furcation tube 290 could be formed of coaxial tubing.

The length of furcation tube 290 may be chosen to extend from the device 210 in the sterile field (e.g., where the patient is) to a location outside of the patient, such as a medical fluid injector, or to a standalone display device, or to some other processing or computing equipment 296 positioned some distance from the patient. The SC connector 294 is adapted to interconnect with an injector (or other signal processing unit) appropriately configured. If signal processing is done within the injector, then the injector display could be utilized to display pressure waveforms and/or to calculate and display P_{d}, Pₚ, and/or P_{d}/Pₚ values. A sensor of a guidewire could also be in communication with computing equipment 296 or the injector.

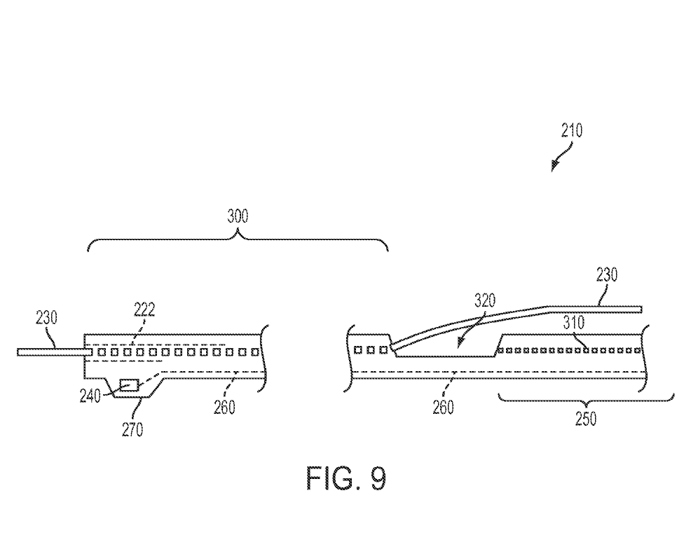

An alternate embodiment would be to construct a distal portion 300 of the sensor delivery device 210 using a dual lumen configuration. An example of such an embodiment is illustrated in FIG. 9. One lumen of the distal portion 300 would accommodate the fiber optic communication channel 260 from the sensor 240 (and from sensor housing 270, in some embodiments). The other lumen (e.g., guidewire lumen 222) would be adapted to slide over the guidewire 230 as shown. The guidewire 230 in such an embodiment would exit from the dual lumen distal portion 300 a certain distance (e.g., about 10-12 inches) back from (e.g., proximal to) the sensor 240 through an opening 320 in the device 210. In some embodiments, a stiffening wire 310 could be placed in the remaining proximal portion of the lumen 222 (that is, the portion of the guidewire lumen 222 in the proximal portion 250 of device 210). The stiffness of the stiffening wire 310 could be varied, for example, to aid a physician in deploying and positioning the device 210 through a catheter and into a particular anatomical (e.g., vascular) structure of interest. The stiffening wire 310 could be part of the dual-lumen device 210, or could be an optional, removable item selected by a physician to obtain the desired amount of stiffness according to some embodiments.

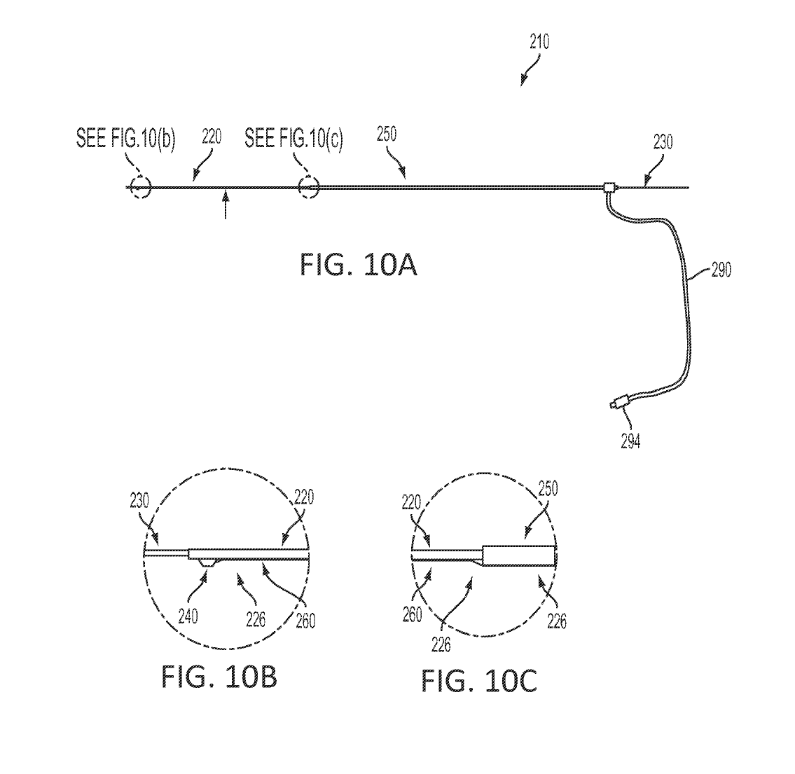

Another alternate embodiment of the invention would be an entirely over-the-wire (OTW) device, substantially as shown in FIG. 10. FIG. 10 illustrates an embodiment in which both the distal sleeve 220 and the proximal portion 250 of sensor delivery device 210 are adapted to slide over a guidewire 230. The guidewire 230 in such an embodiment would not exit from or separate from the device 210 at some point along the length of device 210. Instead, the entire length of the proximal portion 250 of device 210 would slide over the guidewire 230 within a guiding catheter (not shown). The design of the device may incorporate two different sizes of tubes, for example, to form the distal sleeve 220 and proximal portion 250. For example, a smaller diameter thin-walled tube could form the distal sleeve 220, where the sensor 240 resides (optionally, within a sensor housing 270). Back some distance from the location of sensor 240 on the distal sleeve 220, the smaller diameter tube of the distal sleeve 220 would transition into a larger diameter portion (e.g., proximal portion 250), with sufficient clearance between the inner wall of both tubes and the guidewire. Such clearance may provide less friction and sliding resistance while positioning the sensor 240, for example. The larger diameter tube of the proximal portion 250 could be made, for example, from a material with a low coefficient of friction to lower the sliding force. The sensor 240 (and sensor housing 270, where applicable) could be of similar construction to that described above with respect to FIGS. 5A-5D.

FIG. 10 is an example of an embodiment of the invention that illustrates the over-the-wire concept. The larger diameter tubing of the proximal portion 250 could be formed of a single lumen tube or a dual lumen tube. With a single lumen tube, the communication channel 260 (e.g., fiber optic) could be disposed on an outer surface of the proximal portion 250, for example, and could extend toward a connector at a proximal end of the device 210. In embodiments with a dual lumen tube forming the proximal portion 250, the communication channel 260 could extend toward a connector at a proximal end of the device 210 within the second lumen. This could, for example, provide added protection for the communication channel 260 (e.g., fiber optic).

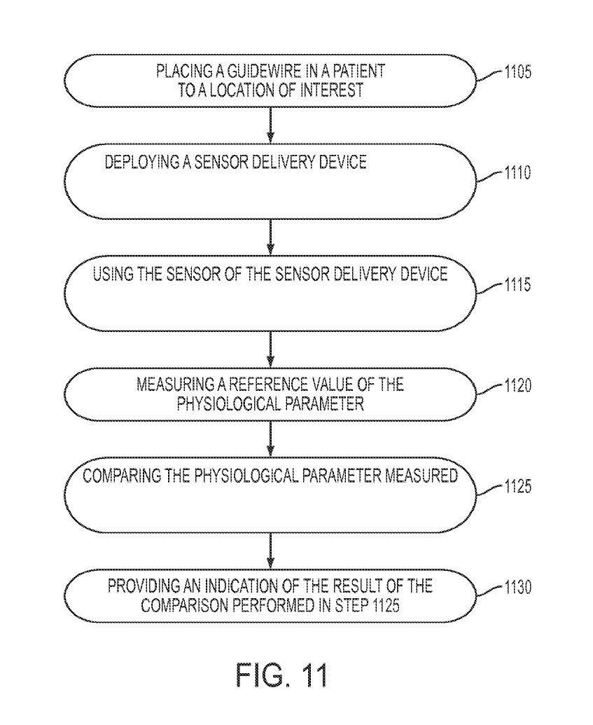

FIG. 11 is a flow diagram showing an exemplary method of using a sensor delivery device. As shown, the method may be used to assess the severity of a stenotic lesion in a patient's vasculature. Step 1105 comprises placing a guidewire in a patient to a location of interest. In some embodiments, this may be a diagnostic guidewire, and a guiding catheter may also be inserted into the patient in conjunction with the guidewire. In some embodiments, the guidewire has an integrated sensor and placing the guidewire in the patient comprises positioning the integrated sensor at or adjacent a location of interest. For example, the integrated sensor of the guidewire may be positioned distal to a location of interest in the patient. Step 1110 comprises deploying a sensor delivery device over the guidewire such that the sensor is positioned at or adjacent a location of interest. In some embodiments, the sensor delivery device will have a sensor, a distal sleeve that slides over the guidewire, and a proximal portion that is used to advance the distal sleeve over the guidewire without having to move the guidewire. In some embodiments, the sensor delivery device is deployed so the sensor of the sensor delivery device is positioned proximal to the location of interest in the patient.

The technique of FIG. 11 also include step 1115, which comprises using the sensor of the sensor delivery device to measure a physiological parameter of interest at or adjacent the location of interest. In some embodiments, the physiological parameter is blood pressure measured proximal to a stenotic lesion. Step 1120 comprises measuring a reference value of the physiological parameter of interest. In some embodiments, this step comprises measuring blood pressure distal to the stenotic lesion. This could be done, for example, with the sensor of the sensor delivery device or a separate blood pressure monitoring apparatus. For example, when the sensor delivery device is used with a guidewire have an integrated sensor, the guidewire sensor may also measure a physiological parameter of interest at or adjacent the location of interest. The guidewire sensor may measure blood pressure distal to the stenotic lesion, e.g., simultaneously with the measurement made by the sensor delivery device sensor. The blood pressure measured by the guidewire sensor may act as the reference pressure for purposes of step 1120. Step 1125 may be an optional step which comprises comparing the physiological parameter of interest measured at the location of interest to the reference value measured in step 1120. In some embodiments, this may comprise calculating a ratio of the two measured values. In one preferred embodiment of the invention, step 1125 comprises calculating P_{d}/Pₚ as the ratio of downstream to upstream blood pressures (e.g., distal to proximal blood pressures). Step 1130 may be an optional step which comprises providing an indication of the result obtained in step 1125. For example, step 1130 may comprise providing a visual indication of the calculated P_{d}/Pₚ value, or may provide other visual cues (e.g., providing a color-coded indication of the severity of a stenotic lesion, such as a red indicator for FFR values less than 0.75, and a green indicator for P_{d}/Pₚ values equal to or greater than 0.75, as possible examples).

It may be desirable, as mentioned above with respect to FIG. 8, to have the sensor delivery device 210 interact with other devices and/or display equipment. For example, a furcation tube 290 and a connector 294 may be used to send the signal (e.g., the measured physiological parameter signal) from sensor 240 to processing device 296. In some embodiments, processing device 296 can also be in communication with a guidewire having a sensor, as discussed further below. Processing device 296 could be, for example, a standalone display monitor to show signal waveforms and/or numerical values of the physiological parameter signal from sensor 240. Processing device 296 may include one or more processors, such as one or more microprocessors, digital signal processors (DSPs), application specific integrated circuits (ASICs), field programmable gate arrays (FPGAs), programmable logic circuitry, or the like, either alone or in any suitable combination.

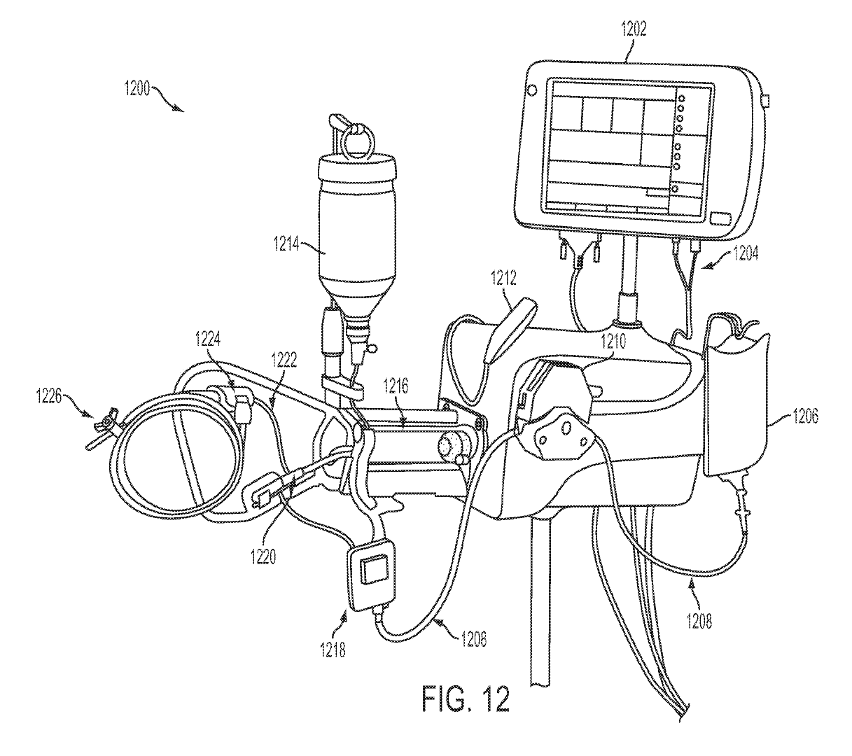

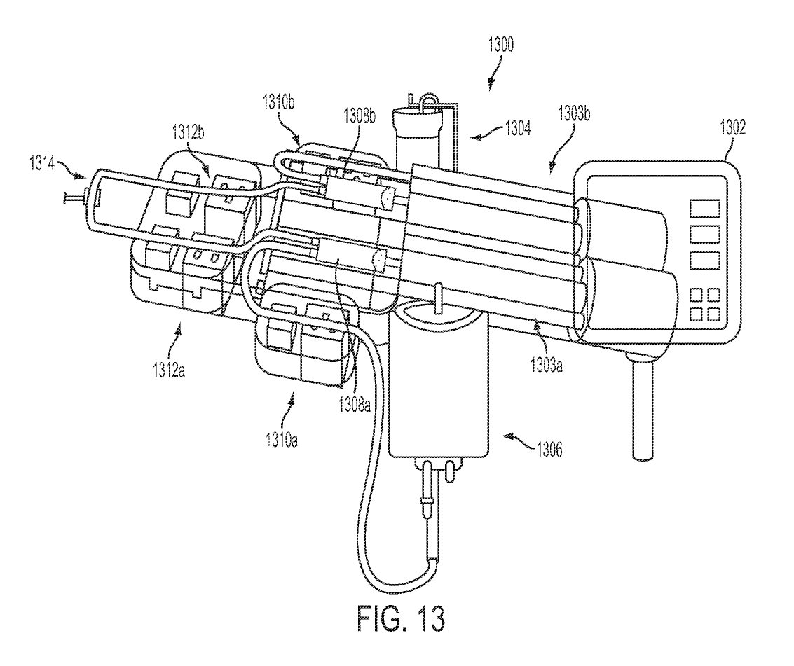

Processing device 296 could include data recording capabilities in some embodiments. In some embodiments, processing device 296 could comprise a medical fluid injection system, such as a powered fluid injector used to inject contrast media and/or saline during certain imaging procedures (e.g., angiography, computed tomography, MRI, ultrasound, etc.). FIGS. 12 and 13 illustrate exemplary powered injection systems which may be used with a sensor delivery device according to various embodiments.

FIG. 12 is a perspective view of one embodiment of a powered injection system 1200 that may be used to perform various functions and, when operable, may be coupled to a physiological sensor delivery device, such as the various embodiments of a sensor delivery device described above, and a guidewire with a sensor, as discussed further below. The powered injection system 1200 shown in FIG. 12 may be used to inject medical fluid, such as contrast media or saline, into a patient within the sterile field during a medical procedure (such as during an angiographic or CT procedure). A physiological sensor delivery device and/or a guidewire with a sensor may be coupled to the system 1200 and used within the sterile field during a patient procedure, according to one embodiment. The system 1200 includes various components, such as a control panel 1202, a hand-controller connection 1204, a hand controller 1212, a fluid reservoir 1206, tubing 1208, a pump 1210, a pressure transducer 1218, a fluid reservoir 1214, an injection syringe 1216, high pressure injection tubing 1222, a valve 1220, an air detector 1224, and a stopcock 1226. In one embodiment, described in more detail below, the fluid reservoir 1206 comprises a container such as, for example, a bag or bottle of diluent (such as saline), the fluid reservoir 1214 comprises a container such as, for example, a bag or bottle of contrast media, and the pump 1210 comprises a peristaltic pump. In other embodiments, the pump 1210 may comprise other forms of pumping devices, such as a syringe, a gear pump, or other form of displacement pump. In some embodiments, the injection syringe 1216 (along with its associated plunger), which is a pumping device, may be replaced with another form of pumping device that delivers high-pressure fluid injections to a patient. An individual pumping device is capable of operating or functioning in different, or multiple, operational modes. For example, a pumping device may be operable to pump fluid when actuated, or driven, to move in a first direction (e.g., forward), while it may also be operable to move in a second direction (e.g., an opposite direction, backward) to carry out certain functions.

The system 1200 of FIG. 12 also shows a hand controller 1212 and an air detector 1224. An operator may use the hand controller 1212 to manually control injection of saline and/or contrast media. The operator may push a first button (not shown) on the hand control 1212 to inject saline, and may push a second button (not shown) to inject contrast, for example. In one embodiment, the operator may push on the contrast button to deliver contrast at a variable flow rate. The harder the operator pushes on the button, the greater the flow rate of contrast media delivered to the patient. Other controllers, such as foot pedal controllers, may also be used. The air detector 1224 is able to detect potential air bubbles or columns within the high-pressure tubing 1222. In one embodiment, the air detector 1224 is an ultrasonic or acoustic-based detector. In other embodiments, the air detector 1224 may use infrared or other detection means (such as optical). If the air detector 1224 detects the presence of air in the high-pressure tubing 1222, it generates a signal that is used to warn the operator and/or halt an injection procedure.

An operator may use the control panel 1202 to view and/or select various parameters and/or protocols to be used during a given procedure. The control panel 1202 may be used to display information to an operator about the status of the equipment and/or the patient. The pump 1210 may be used to pump saline from the bag into the patient via the saline tubing 1208, the valve 1220, and the high-pressure tubing 1222. In one embodiment, the valve 1220 comprises a spring-based spool valve, as is known in the art. In one embodiment, the valve 1220 comprises an elastomeric-based valve.

In one embodiment, the syringe 1216 is used to draw contrast from the reservoir 1214 into the syringe 1216, and to inject contrast from the syringe 1216 into the patient via the valve 1220 and high-pressure tubing 1222. In one embodiment, the syringe 1216 is a self-purging syringe that has one port for filling of contrast and purging of air, and a second port for injection of contrast.

The valve 1220 may be used to control coupling between input ports to the valve 1220 and an output port. In one embodiment, the valve includes two input ports, one which is coupled to the contrast fluid line and another which is coupled to the saline fluid line. The saline fluid line also includes a pressure transducer 1218 for providing a signal representative of patient blood pressure, for example.

The stopcock 1226 regulates the flow of fluids to the patient. In one embodiment, the valve 1220 allows either the saline line or the contrast line to be coupled to the patient (high-pressure tubing) line 1222. When the syringe 1216 is used to inject contrast media, for example, the valve 1220 may allow the contrast media to flow to the patient line 1222 while blocking the flow of saline to the patient line 1222. Valve 1220 may operate such that the pressure transducer 1218 may also be blocked or isolated from the patient line 1222 during high-pressure injections, for example, to protect the transducer 1218 from high injection pressures that may accompany a contrast injection. When there is no injection of contrast from the syringe 1216, the valve 1220 may operate to block the contrast line from the patient line 1222, while opening the fluid connection between the saline line (tubing) 1208 and the patient line 1222. In this state, the pump 1210 is capable of injecting saline into the patient, and the pressure transducer 1218 is also capable of monitoring hemodynamic signals coming from the patient via the patient line 1222 and generating representative signals based upon the measured pressures.

As noted above, the system 1200 of FIG. 12 may be adapted to be coupled to a physiological sensor delivery device and/or a guidewire having a sensor. System 1200 may, for example, be adapted to receive the physiological signal generated by the sensor 240 of device 210 and/or sensor 31 of a guidewire 30. In embodiments where the received physiological signal is a pressure signal measured downstream of a stenotic lesion (e.g., P_{d}), system 1200 may facilitate calculation of a P_{d}/Pₚ, for example, since Pₚ may already be provided by pressure transducer 1218 of system 1200. Additionally or alternatively, system 1200 may receive a signal from sensor 240 of device 210 indicative of blood pressure at one location (e.g., proximal to a stenotic lesion) and another signal from a guidewire sensor indicative of blood pressure at a different location (e.g., distal to the stenotic lesion). System 1200 may calculate a characteristic of interest, such as P_{d}/Pₚ, based on the received signals. System 1200 may or may not also use an additional proximal pressure measurement provided by the pressure transducer of system 1200 in performing the calculation. A visual or graphical display of the calculated P_{d}/Pₚ values could be presented to an operator via control panel 1202, for example. Since instantaneous values of Pₚ and P_{d} are available in such an arrangement, the timing effects and associated errors would not pose a problem—simultaneous measurement of Pₚ and P_{d} would reduce or eliminate such errors. In addition, time averaging or other signal processing could be employed by system 1200 to produce mathematical variants of the P_{d}/Pₚ calculation (e.g., mean, max, min, etc.). Alternately, a time-varying display or plot of the calculated P_{d}/Pₚ values could be displayed as a waveform (e.g., as a function of time).

FIG. 13 is a perspective view of another embodiment of a powered injection system 1300 that may be used to perform various functions and, when operable, may be coupled to a physiological sensor delivery device and/or a guidewire with a sensor, such as the embodiments described above. The powered injection system 1300 shown in FIG. 13 may be used to inject medical fluid, such as contrast media or saline, into a patient within the sterile field during a medical procedure (such as during an angiographic or CT procedure). A physiological sensor delivery device may be coupled to the system 1300 and used within the sterile field during a patient procedure, according to one embodiment.

The system 1300 of FIG. 13 is a dual-syringe system that includes a control panel 1302 and two motor/actuator assemblies 1303a and 1303b. Each motor drives one of the linear actuators in the assemblies 1303a, 1303b. Each linear actuator drives a plunger of one syringe 1308a or 1308b. An individual plunger moves within the syringe barrel of the syringe 1308a or 1308b in either a forward or rearward direction. When moving in a forward direction, the plunger injects liquid into the patient line or purges air out of the syringe and into a liquid container (e.g., bottle). When moving in a rearward direction, the plunger fills liquid into the syringe 1308a, 1308b from a liquid container. FIG. 13 shows examples of two such liquid containers 1304 and 1306. In one embodiment, the container 1304 is a bag or bottle containing contrast agent, and the container 1306 is a bag or bottle containing diluent, such as saline. In other embodiments, the syringes 1308a, 13808b (along with associated plungers), which are each pumping devices, may either separately or together comprise another form of pumping device that is capable of injecting fluids at appropriate flow rates/pressures/etc., such as, for example, a peristaltic pump or another form of displacement pump. An individual pumping device is capable of operating or functioning in different, or multiple, operational modes. For example, a pumping device may be operable to pump fluid when actuated, or driven, to move in a first direction (e.g., forward), while it may also be operable to move in a second direction (e.g., an opposite direction, backward) to carry out certain functions. Multiple sets of pinch valve/air detect assemblies are shown in FIG. 13. One pinch valve/air detect assembly 1310a is coupled between the liquid container 1306 and a syringe input port of the syringe 1308a, and a second pinch valve/air detect assembly 1312a is coupled between a syringe output port of the syringe 1308a and the patient connection. A third pinch valve/air detect assembly 1310b is coupled between the liquid container 1304 and a syringe input port of the syringe 1308b, and a fourth pinch valve/air detect assembly 1312b is coupled between a syringe output port of the syringe 1308b and the patient connection. In the embodiment shown in FIG. 13, each syringe 1308a, 1308b is a dual-port syringe. Fluid flows and is drawn into the syringe 1308a or 1308b from a container via the syringe input port, and fluid flows out of and is injected from the syringe 1308a or 1308b via the syringe output port.

Each pinch valve is a pinch valve/air detect assembly 1310a, 1310b, 1312a, 1312b may be opened or closed by the system 1300 to control the fluid connections leading to or away from each of the syringes 1308a, 1308b. The air detect sensors in the assemblies 1310a, 1310b, 1312a, 1312b may be optical, acoustic, or other form of sensor. These sensors help detect air that may be present in the fluid connections leading to or away from the syringes 1308a, 1308b. When one or more of these sensors generates a signal indicating that air may be present in a fluid line, the system 1300 may warn the user or terminate an injection procedure. The use of multiple pinch valves within the system 1300 allows the system 1300 automatically, or through user interaction, to selectively control the flow of fluid into or out of the syringes 1308a, 1308b by opening or closing fluid tubing. In one embodiment, the system 1300 controls each of the pinch valves. The use of multiple air-detect sensors helps improve the overall safety of the system 1300 by detecting possibly air (e.g., columns, bubbles) within fluid (in the tubing) leading to or away from the syringes 1308a, 1308b. Signals from the air detectors are sent to and processed by the system 1300, such that the system 1300 may, for example, provide a warning, or terminate an injection procedure, if air is detected. In the example of FIG. 13, the fluid tubing first flows through a pinch valve and then flows through an air detector within the assemblies 1310a, 1310b, 1312a, 1312b. In other embodiments, other configurations, ordering, and the like may be used for the pinch valves and air detectors within these assemblies. Moreover, other types of valves may be substituted for the pinch valves.

An operator may use the control panel 1302 to initialize, or setup, the injection system 1300 for one or more injection procedures, and may further use the control panel 1302 to configure one or more parameters (e.g., flow rate, volume of fluid to be delivered, pressure limit, rise time) of an individual injection procedure. The operator may also use the panel 1302 to pause, resume, or end an injection procedure and begin a new procedure. The control panel also displays various injection-related information to the operator, such as flow rate, volume, pressure, rise time, procedure type, fluid information, and patient information. In one embodiment, the control panel 1302 may be connected to a patient table, while being electrically coupled to the main injector of the system 1300. In this embodiment, the operator may manually move the control panel 1302 to a desirable location, while still having access to all functionality provided by the panel 1302.

The system of FIG. 13 also includes a valve 1314 coupled to both output lines coming from the syringes 1308a and 1308b. Each syringe output provides fluid injected through tubing that passes through a pinch valve/air detect assembly 1312a or 1312b and that then leads to an input of the valve 1314. In one embodiment, one fluid line to the valve 1314 also includes a pressure transducer. The valve output port of the valve 1314 is coupled to a high-pressure tubing line, which is used to direct fluid to the patient. In one embodiment, the valve 1314 is made of a flexible material, such as an elastomeric material. The valve 1314 allows one of the fluid lines (e.g., the contrast line or the saline line) to be coupled to the patient (high-pressure tubing) line. When saline and contrast are contained within the syringes 1308a and 1308b, respectively, the valve 1314 allows the contrast media to flow from the syringe 1308b to the patient line (assuming the pinch valve in the assembly 1312b is open and there has been no air detected), but blocks the flow of saline from the syringe 1308a to the patient line. The pressure transducer coupled to the saline line (according to one embodiment) is also blocked from the patient line, thereby protecting the transducer from high injection pressures that may accompany a contrast injection. When there is no injection of contrast from the syringe 1308b, the valve 1314 blocks the contrast line from the patient line, but allows a connection between the saline line from the syringe 1306 to the patient line. The syringe 1308a is capable of injecting saline into the patient (assuming the pinch valve in the assembly 1312a is open and there has been no air detected), and the pressure transducer is also capable of monitoring hemodynamic signals coming from the patient via the patient line, and generating representative electronic signals based upon the measured pressures that can be processed by the system 1300.

In one embodiment, a secondary control panel (not shown) provides a subset of functions provided by the main panel 1302. This secondary control panel (also referred to herein as the “small” control panel) may be coupled to the injector within the system 1300. In one scenario, the operator may use the small panel to manage injector setup. The small panel may display guided setup instructions that aid in this process. The small panel may also display certain error and troubleshooting information to assist the operator. For example, the small panel may warn the operator of low contrast or saline fluid levels in the liquid reservoirs and/or syringes.

As with the system 1200 of FIG. 12, system 1300 of FIG. 13 may be adapted to be coupled to a physiological sensor delivery device and/or a guidewire with a sensor according to certain embodiments of the invention. System 1300 may, for example, be adapted to receive the physiological signal generated by the sensor 240 of device 210 and/or the physiological signal generated by sensor 31 of the guidewire 30. Processing of the physiological signals from the sensors may be performed within the injection system 1200 or 1300, for example. Signal conditioning and/or processing may, for example, be performed by a circuit board or card that may be an add-on feature to system 1200 or 1300. Such a signal conditioning board or card may process a “raw” signal from the sensor(s) and convert the signal into a standard analog and/or digital signal, which can be used by processors of the injector system, according to some embodiments. The processed signal may enable injector system 1200 or 1300 to display the signal data (e.g., as pressure waveforms), and/or perform algorithms and/or calculations and display the results.

In embodiments where a received physiological signal is a pressure signal measured downstream of a stenotic lesion (e.g., P_{d}), system 1300 may facilitate calculation of P_{d}/Pₚ, for example, since Pₚ may already be provided by the pressure transducer of system 1300. Additionally or alternatively, system 1300 may receive a signal from sensor 240 of device 210 indicative of blood pressure at one location (e.g., proximal to a stenotic lesion) and another signal from a guidewire sensor indicative of blood pressure at a different location (e.g., distal to the stenotic lesion). System 1300 may calculate a characteristic of interest, such as P_{d}/Pₚ, based on the received signals. System 1300 may or may not also use an additional proximal pressure measurement provided by the pressure transducer of system 1300 in performing the calculation. A visual or graphical display of the calculated P_{d}/Pₚ values, for example, could be presented to an operator via control panel 1302, for example, or via a small control panel (not shown) having a subset of the functions provided by control panel 1302. In addition, time averaging or other signal processing could be employed by system 1300 to produce mathematical variants of the P_{d}/Pₚ calculation (e.g., mean, max, min, etc.).

In some embodiments, a method may include basing a therapy decision on the calculated P_{d}/Pₚ value, e.g., if the calculated P_{d}/Pₚ is less than 0.75, an interventional therapy is recommended and/or performed. In some embodiments, an interventional therapy device may be deployed by withdrawing sensor delivery device 210, and using the same guidewire 230 to deploy the interventional therapy device.

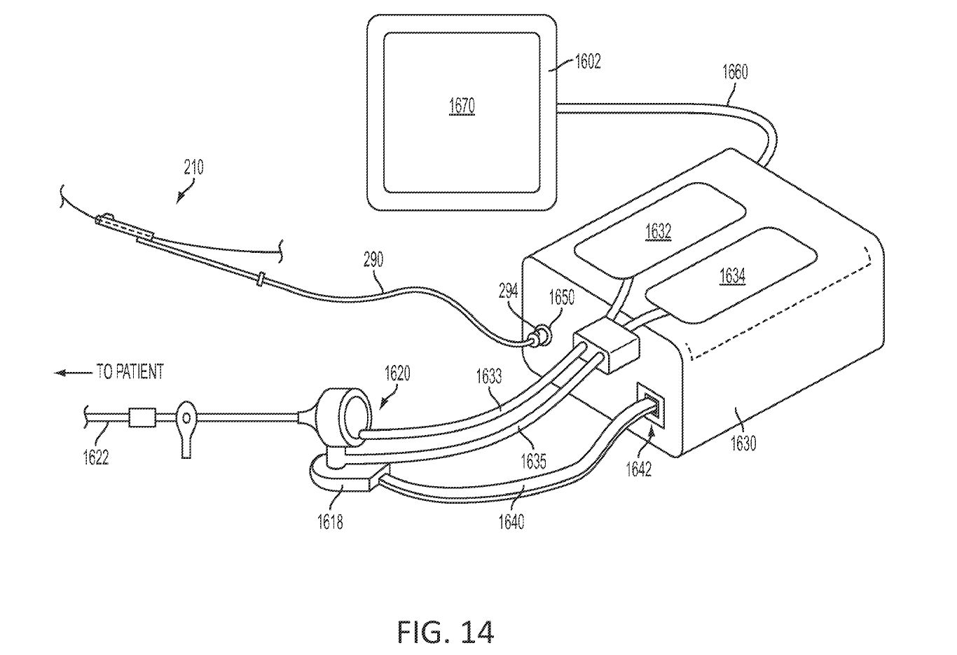

FIG. 14 is a perspective view of a powered injection system adapted to be coupled to a physiological sensor delivery device according to certain embodiments. FIG. 14 shows a sensor delivery device 210 connected to a powered injection system 1630 via furcation tube 290 and connector 294. Injection system 1630 is adapted to receive a physiological measurement signal (e.g., blood pressure) from device 210 via input port 1650. In some embodiments, the signal is an optical signal, and connector 294 is an SC fiber optic connector adapted to mate with port 1650 to receive the optical signal.

As shown in FIG. 14, system 1630 has 2 fluid containers 1632, 1634, which are adapted to deliver fluid through lines 1633 and 1635. Fluid in line 1633 (e.g., contrast solution) may be delivered at significantly higher pressures than fluid in line 1635 (e.g., saline solution), for example. Valve 1620 may be used to control coupling between input ports to the valve 1620 and to an output port which ultimately leads to a patient via patient line 1622. In one embodiment, valve 1620 includes two input ports, one which is coupled to a contrast fluid line 1633 and another which is coupled to a saline fluid line 1635. The saline fluid line is also coupled to a pressure transducer 1618 for providing a signal representative of patient blood pressure, for example. The signal from pressure transducer 1618 may be communicated to system 1630 via communication path 1640 and connector 1642, or via other equivalent means (e.g., infrared, optical, etc.).

In one embodiment, the valve 1620 allows either the saline line or the contrast line to be coupled to the patient (high-pressure tubing) line 1622. When the system 1630 is injecting contrast media, for example, the valve 1620 may allow the contrast media to flow to the patient line 1622 while blocking the flow of saline to the patient line 1622. Valve 1620 may operate such that the pressure transducer 1618 may also be blocked or isolated from the patient line 1622 during high-pressure injections, for example, to protect the transducer 1618 from high injection pressures that may accompany a contrast injection. When there is no injection of contrast from the system 1630, the valve 1620 may operate to block the contrast line from the patient line 1622, while opening the fluid connection between the saline line (tubing) 1635 and the patient line 1622. In this state, the system 1630 may be capable of injecting saline into the patient, while the pressure transducer 1618 is capable of monitoring hemodynamic signals coming from the patient via the patient line 1622, and generating representative signals based upon the measured pressures.

FIG. 14 shows control panel 1602 connected to injection system 1630 via communication path 1660. An operator may interact with system 1630 via control panel 1602 (or via a secondary panel, if available) to review and/or modify injection parameters, for example. In some embodiments, system 1630 is adapted to receive pressure signals simultaneously from pressure transducer 1618 and from device 210, representative of upstream and downstream pressures (e.g., Pₚ, P_{d}), respectively. In other embodiments, system 1630 is adapted to receive pressure signals simultaneously from a guidewire pressure sensor and from device 210, representative of distal and proximal pressures (e.g., P_{d}, Pₚ), respectively. In yet other embodiments, system 1630 is adapted to receive pressure signals simultaneously from a guidewire pressure sensor, from device 210, and from pressure transducer 1618. In either embodiment, system 1630 receives two or more pressure signals (e.g., P_{d} and Pₚ) substantially simultaneously, compares the two or more signals (e.g., calculates P_{d}/Pₚ), and provides an indication of the result of the comparison to an operator via a display screen 1670 of control panel 1602. As noted above, the indication of the result of the comparison may take a number of different forms, including numerical, graphical, time plots, etc. The indication may be of the pass/fail variety, for example, indicating one color-coded pattern (e.g., a RED icon) for an P_{d}/Pₚ value below a certain value (e.g., 0.75), and/or a different color-coded pattern (e.g., a GREEN icon) for an P_{d}/Pₚ value at or above a certain value (e.g., 0.75). The indication may also be an audible alarm according to some embodiments of the invention.

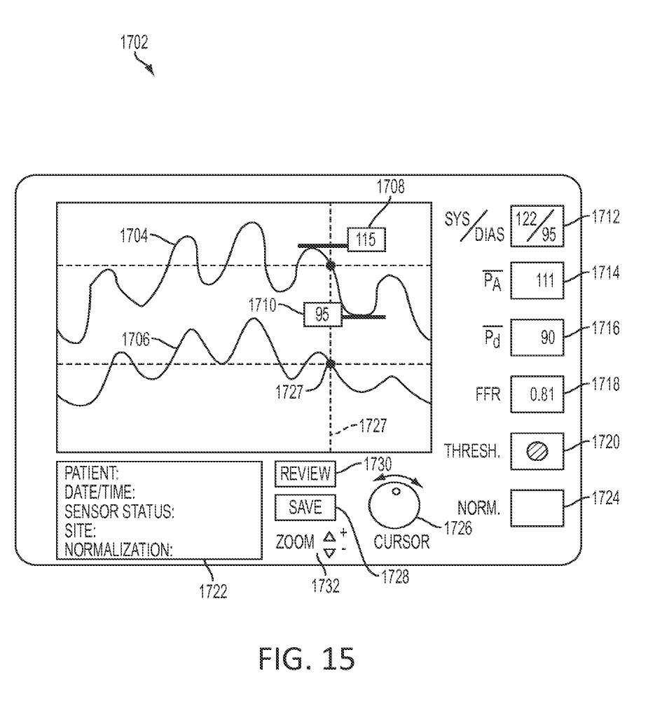

FIG. 15 is an idealized view of information that may be displayed (e.g., via an interactive graphical user interface, or “GUI interface”) to an operator, according to certain embodiments of the invention. FIG. 15 shows a GUI screen that may be displayed either via a control panel that is unique to the sensor delivery device 210, or via a control panel of a device adapted for use with device 210, such as the powered fluid injection systems described above with respect to FIGS. 12, 13, and 14. (The GUI interface could be implemented in software such that a user might see a very similar screen regardless of whether a stand-alone display device or an integrated injector system was being used, according to various embodiments of the invention.)

In FIG. 15, screen 1702 is adapted to display data in various forms (e.g., waveform data, numerical data, calculated values, patient information, device status information, etc.). For example, in a preferred embodiment of the invention useful for making P_{d}/and Pₚ measurements, blood pressure waveforms may be displayed as a function of time for both proximal pressure, Pₚ(t) 1704, and distal pressure, P_{d}(t) 1706. In some embodiments, systolic and diastolic blood pressure measurements may be superimposed on the time plot for the proximal (e.g., aortic) pressure waveform, as shown at 1708 and 1710, respectively, and/or may be calculated as average values and displayed substantially as shown at 1712. Similarly, average values for proximal pressure 1704 and distal pressure 1706 may be calculated (e.g., these could be time-weighted averages, moving averages, etc.) and displayed as shown at 1714 and 1716, respectively. A calculation of P_{d}/Pₚ based on proximal pressure 1704 and distal pressure 1706 may also be calculated and displayed as shown at 1718, for example, and the values used for Pₚ and P_{d} could be averages or other forms of statistical or numerical representation), according to some embodiments of the invention. Further, some embodiments may include a feature to alert an operator to an P_{d}/Pₚ value that lies outside of a normal range (e.g., less than 0.75) to indicate, for example, that some other action should be taken (e.g., select and perform an interventional therapy). This could be a visual cue (such as a colored light, as shown at 1720), or could be an audible cue (such as an alarm sound, for example).

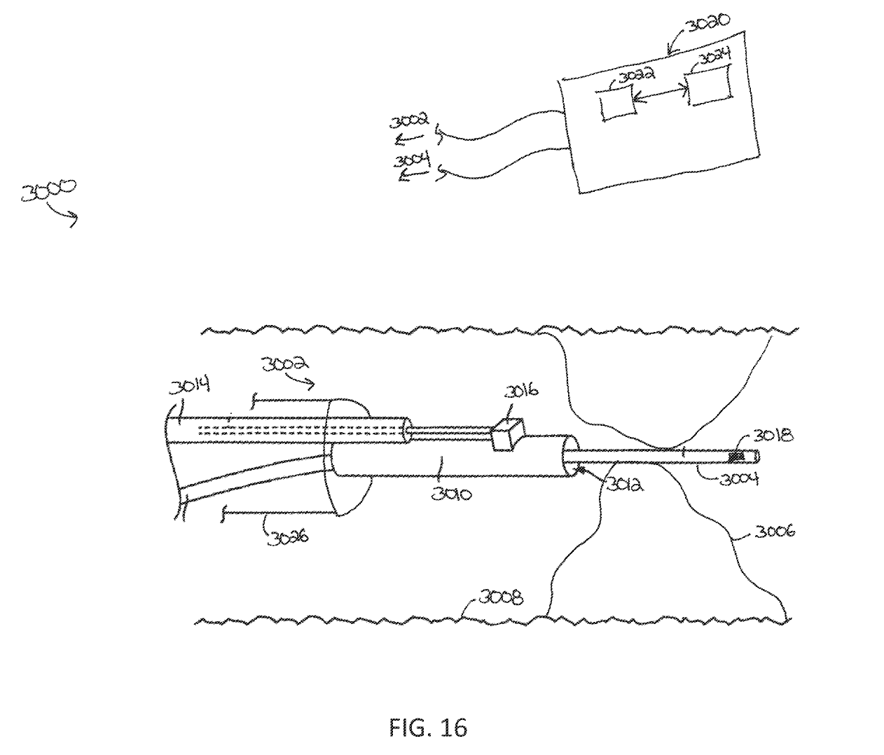

The screen 1702 of FIG. 15 shows various additional features which may be (optionally or alternately) incorporated in various embodiments. Status area 1722, for example, may provide information about the patient, date/time, the site within a particular patient, the status of the sensor, and an indication of whether the sensor signal has been “normalized” to another pressure monitoring signal. A normalization button 1724 may be included in some embodiments, and could be used, for example, to normalize the pressure signal from a sensor of sensor delivery device 210. Normalization might be done during a procedure in which a P_{d} or Pₚ measurement is desired (e.g., to assess the severity of a stenosis). When a sensor of sensor delivery device 210 is positioned upstream of the stenosis, the measured pressure using the sensor should be equal to the proximal pressure measured using normal blood pressure monitoring equipment (e.g., via the pressure transducer 1618 of the injection system shown in FIG. 16, for example). In one embodiment, an operator would position the sensor 240 of sensor delivery device 210 upstream of a location of interest and press the normalization button 1724 of screen 1702, which could then automatically adjust or calibrate the pressure signal from sensor 240 to match the proximal pressure measured using normal blood pressure monitoring equipment.

The screen 1702 of FIG. 15 may also include navigational features, in some embodiments, which may allow an operator to view and record information that may be of interest. For example, a cursor button 1726 may allow an operator to position a marker or cursor 1727 to a point of interest on the waveforms 1704, 1706, which could provide instantaneous measured values of Pₚ(t) 1704 and P_{d}(t) 1706 at a selected point in time. In some embodiments, an operator may elect to save the cursored data by pressing a “save” button 1728, which could save the highlighted data for review at a later point in time. A review button 1730 may be provided for this purpose in some embodiments, allowing a user to compare previous historical measurements to current ones and use this information to make diagnostic and therapeutic decisions. In some embodiments, it may be desirable to include a “zoom” feature, for example, to analyze the data. For example, an operator may wish to zoom in (e.g., via the +arrow of zoom 1732) to look more closely at certain data, or may instead wish to zoom out (e.g., via the −arrow of zoom 1732) to evaluate overall trends, for example.

Any of the various embodiments of sensor delivery devices, processors, injection system, and interfaces described herein may be used with a guidewire having a sensor. In such embodiments, the guidewire sensor can provide a physiological measurement that can be used in conjunction with a physiological measurement obtained by a sensor of a sensor delivery device to provide an assessment of a location of interest within a patient.

In some embodiments, a pressure sensory device is positioned over a guidewire having a pressure sensor, sometimes referred to as a pressure sensing guidewire. Such a guidewire can have a pressure sensor embedded within the guidewire itself. In such embodiments, the pressure sensing guidewire can be deployed across a stenotic lesion so the sensing element is on the distal side of the lesion and the distal blood pressure is recorded via the guidewire sensor. The pressure gradient across the stenosis and the resulting P_{d}/Pₚ value could then be calculated using this information.

Some embodiments include a system with a guidewire having a distal portion and a proximal portion opposite the distal portion. The guidewire may have an integrated sensor in the distal portion. The system also includes a sensor delivery device having a sensor, a distal sleeve, and a proximal portion, the distal sleeve configured to slidably receive the guidewire.

Certain embodiments of the system include a processor configured to receive a first signal (e.g., representative of blood pressure) measured distally of a location of interest in a patient from the sensor of the guidewire, and receive a second signal (e.g., representative of blood pressure) measured proximally of the location of interest from the sensor of the sensor delivery device. The processor may be configured to provide an assessment of the location of interest based on a comparison of the first signal and the second signal. For example, the assessment can include a calculation of a ratio of the first signal to the second signal. In a specific example, the assessment can include a calculation of FFR.

Embodiments of the invention also include methods of positioning sensors within a patient. Such a method can include the steps of positioning a sensor carried by a guidewire distally of a location of interest in a patient, advancing a sensor delivery device over the guidewire and positioning a sensor of the sensor delivery device proximal to the location of interest in the patient, and comparing a signal generated by the sensor carried by the guidewire to a signal generated by the sensor of the sensor delivery device and determining therefrom a characteristic of the location of interest.

In a specific embodiment, the method can include positioning a sensor contained within a guidewire distally of a lesion in a blood vessel of a patient, the sensor contained within the guidewire being configured to generate a first signal representative of fluid pressure. The method can also include the step of advancing a sensor delivery device having a sensor, a distal sleeve, and a proximal portion distally over the guidewire and positioning the sensor of the sensor delivery device proximal to the lesion, the sensor of the sensor delivery device being configured to generate a second signal representative of fluid pressure. The method can also include the step of providing an assessment of the location of interest based on a ratio of the first signal to the second signal.

FIG. 16 is a perspective view of an example system 3000 utilizing a sensor delivery device 3002 and guidewire 3004 for measuring a characteristic of a location of interest in a patient. In the example shown in FIG. 16, sensor delivery device 3002 and guidewire 3004 are deployed to measure a characteristic of a stenotic lesion 3006 within a blood vessel 3008 which could be, for example, a coronary artery of a patient. While the sensor delivery device 3002 can have any configuration as described herein, the delivery device is illustrated as having a distal sleeve 3010 defining a guidewire lumen 3012 for slidably receiving guidewire 3004 and a proximal portion 3014. Sensor delivery device 3002 has a sensor 3016. In addition, guidewire 3004 carries a separate sensor 3018 in a distal portion of the guidewire. Sensor 3016 of sensor delivery device 3002 and sensor 3018 of guidewire 3004 are illustrated as being communicatively coupled to an external computing device 3020 located outside of the body of the patient. External computing device 3020 includes a processor 3022 and a memory 3024. In some examples, external computing device may be a fluid injection system that is configured to inject pressurized medical fluid (e.g., contrast media and/or saline) into the body of the patient, although the disclosure is not limited to such an example computing device.

To characterize the stenotic lesion 3006 in FIG. 16, a clinician can insert guidewire 3004 carrying sensor 3018 into the vascular structure of the patient. The clinician may first insert a guiding catheter 3026 into the blood vessel 3008 of the patient and then advance the guidewire through the guiding catheter. The clinician may advance guidewire 3004 until sensor 3018 is positioned distally of the lesion, as illustrated in FIG. 16. Subsequently, the clinician may position sensor 3016 of sensor delivery device 3002 within blood vessel 3008. The clinician may thread distal sleeve 3010 onto a proximal portion of guidewire 3004 such that guidewire lumen 3012 slides over guidewire 3004. The clinician can advance sensor 3016 by moving the proximal portion 3014 unit the sensor is positioned proximally of lesion 3006, as illustrated in FIG. 16. Once suitably positioned, sensor 3018 of guidewire 3004 can generate a signal representative of blood pressure on the distal (e.g., downstream) side, P_{d}, of stenotic lesion 3006 and sensor 3016 of sensor delivery device 3002 can generate another signal representative of blood pressure on the proximal (e.g., upstream) side, Pₚ, of the stenotic lesion.

Processor 3022 of computing device 3020 is configured to receive the signal generated by sensor 3016 of sensor delivery device 3002 and also the signal generated by sensor 3018 of guidewire 3004. Processor 3022 may compare the signals, e.g., with reference to instructions stored in memory 3024, store data representative of the signals, or perform other processing tasks. Processor 3022 may include one or more processors, such as one or more microprocessors, digital signal processors (DSPs), application specific integrated circuits (ASICs), field programmable gate arrays (FPGAs), programmable logic circuitry, or the like, either alone or in any suitable combination. When computing device 3020 is implemented as a fluid injection system, processor 3022 may perform additional tasks associated with the operation and management of a fluid injection procedure. For example, processor 3022 may receive electrical signals from input devices, such as a remote control or control panel, and provide electrical signals to output devices, such as a fluid injector, a motor, a display, and the like.

As one example of the tasks processor 3022 may perform, the processor may compare the signal received from sensor 3016 of sensor delivery device 3002 with the signal received from sensor 3018 of guidewire 3004. Processor 3022 may also determine a characteristic of stenotic lesion 3006 based on the comparison. For example, processor 3022 may determine a P_{d}/Pₚ value for stenotic lesion 3006 based on the comparison of the signals. To determine the P_{d}/Pₐ value for the stenotic lesion, processor 3022 may determine a measured distal pressure, P_{d}, based on the signal received from sensor 3018 of guidewire 3004 and information (e.g., calibration information) stored in memory 3024. Processor 3022 may further determine a measured proximal pressure, Pₚ, based on the signal received from sensor 3016 of sensor delivery device 3002 and information (e.g., calibration information) stored in memory 3024. Processor 3022 can determine P_{d}/Pₚ by calculating a ratio of the measured distal pressure to the measured proximal pressure, P_{d}/Pₚ. Processor 3022 may store the determined characteristic (e.g., P_{d}/Pₚ) in memory 3024, control a display communicatively coupled to the processor to display the P_{d}/Pₚ value or an indication thereof, and/or perform other suitable tasks.

In general, memory 3024 stores instructions and related data that, when executed by processor 3022, cause system 3000 and processor 3024 to perform the functions attributed to them in this disclosure. Memory 3024 may be one or more computer-readable storage medium, such as one or more non-transitory computer-readable storage medium, containing instructions. Computer readable storage media may include random access memory (RAM), read only memory (ROM), programmable read only memory (PROM), erasable programmable read only memory (EPROM), electronically erasable programmable read only memory (EEPROM), flash memory, a CD-ROM, or other computer readable media.

For patients with more complex pathological conditions, additional pressure date beyond the pressure data generated by sensor 3016 of sensor delivery device 3002 and sensor 3018 of guidewire 3004 may be useful to accurately characterize a location of interest in the patient. For example, to obtain accurate P_{d}/Pₚ measurements in patients with multiple stenotic lesions, such as tandem lesions in series, a more complex approach may be needed to accurately determine the P_{d}/Pₚ value of each individual lesion.

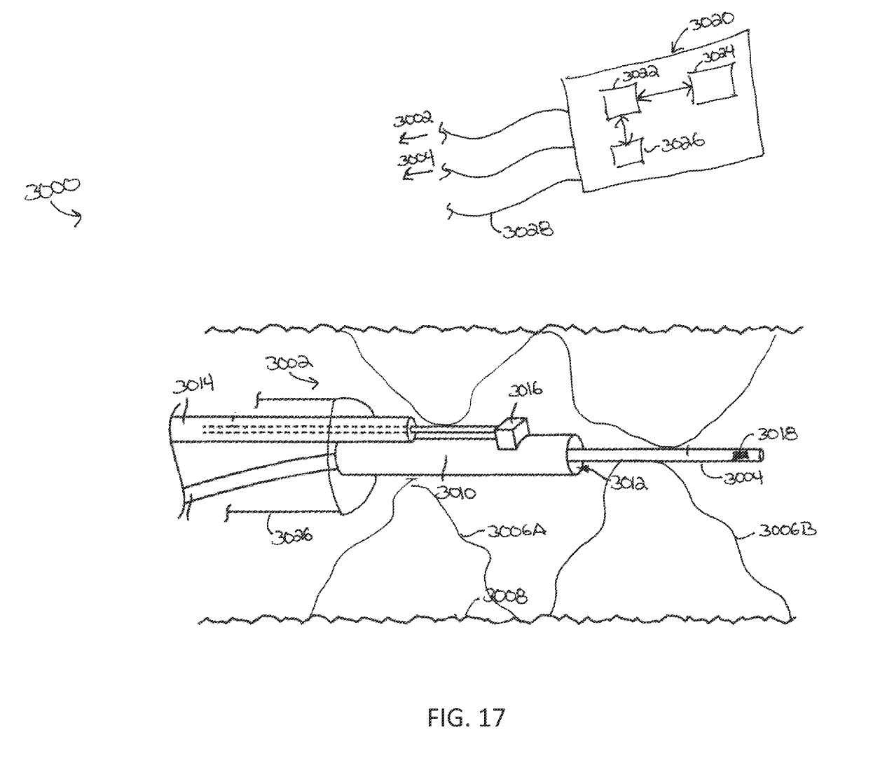

FIG. 17 is a perspective view of an example implementation of system 3000 described above with respect to FIG. 16, where the system is implemented to characterize multiple locations of interest in the patient. In particular, in the example of FIG. 17, system 3000 is illustrated as being implemented to characterize tandem stenotic lesions 3006A and 3006B within blood vessel 3008. Computing device 3020 in the example of FIG. 17 is illustrated as being a fluid injection system that is configured to inject pressurized medical fluid (e.g., contrast media and/or saline) into the body of the patient. Fluid injection system 3020 includes a pressure transducer 3026 that is in fluid communication with a patient line 3028 extending from the injection system 3020 to the patient. Pressure transducer 3026 is communicatively coupled to processor 3026. In operation, pressure transducer 3026 can measure a hemodynamic pressure or other blood pressure of the patient via a column of fluid extending from the patient (e.g., from blood vessel 3008) and through patient line 3028 back to the fluid injection system 3020.

To characterize stenotic lesions 3006A and 3008B, system 3000 may make three pressure measurements (e.g., three simultaneous pressure measurements): a first pressure measurement distal to stenotic lesion 3006B, a second pressure measurement between stenotic lesions 3006A and 3006B, and a third pressure measurement proximal to stenotic lesion 3006A. To make the pressure measurements, a clinician can insert guidewire 3004 carrying sensor 3018 into the vascular structure of the patient. The clinician may first insert a guiding catheter 3026 into the blood vessel 3008 of the patient and then advance the guidewire through the guiding catheter. The clinician may advance guidewire 3004 until sensor 3018 is positioned distally of the lesion 3006B, as illustrated in FIG. 17. Subsequently, the clinician may position sensor 3016 of sensor delivery device 3002 within blood vessel 3008. The clinician may thread distal sleeve 3010 onto a proximal portion of guidewire 3004 such that guidewire lumen 3012 slides over guidewire 3004. The clinician can advance sensor 3016 by moving the proximal portion 3014 unit the sensor is positioned between lesion 3006A and 3006B, as illustrated in FIG. 17. When so positioned, sensor 3016 is proximal to lesion 3006B and distal to 3006A. Pressure transducer 3026 may provide the third pressure measurement indicative of blood pressure proximal to lesion 3006A via the column of fluid extending proximally from lesion 3006A back to the pressure transducer. Alternatively, a second intravascular sensor delivery device and/or a second guidewire carrying a third sensor can be inserted into blood vessel 3008 and positioned so that the sensor measures blood pressure proximally to lesion 3006A.

Once the sensors of system 3000 are suitably positioned, sensor 3018 of guidewire 3004 can generate a signal representative of blood pressure on the distal (e.g., downstream) side of stenotic lesion 3006B, sensor 3016 of sensor delivery device 3002 can generate another signal representative of blood pressure between stenotic lesions 3006A and 3006B, and pressure transducer 3026 can generate a signal representative of blood pressure proximal to stenotic lesion 3006A. Processor 3022 is configured to receive the signal generated by sensor 3016 of sensor delivery device 3002, the signal generated by sensor 3018 of guidewire 3004, and the signal generated by pressure transducer 3026 of fluid injection system 3020. Processor 3022 may compare the signals, e.g., with reference to instructions stored in memory 3024, store data representative of the signals, or perform other processing tasks.

For example, processor 3022 may compare the signal received from sensor 3016 of sensor delivery device 3002 with the signal received from sensor 3018 of guidewire 3004 and the signal received from pressure transducer 3026 of fluid injection system 3020. Processor 3022 may also determine a characteristic of stenotic lesion 3006A and a characteristic of stenotic lesion 3006B based on the comparison. For example, processor 3022 may determine a P_{d}/Pₚ value for stenotic lesion 3006 and also a P_{d}/Pₚ value for stenotic lesion 3006B based on the comparison of the signals.

In some embodiments, to determine a P_{d}/Pₚ value for the stenotic lesions, processor 3022 may determine a measured distal pressure, P_{d}, based on the signal received from sensor 3018 of guidewire 3004 and information (e.g., calibration information) stored in memory 3024. Processor 3022 may further determine a measured middle pressure, Pₘ, based on the signal received from sensor 3016 of sensor delivery device 3002 and information (e.g., calibration information) stored in memory 3024. In addition, processor 3022 may determine a measured proximal pressure, Pₚ, based on the signal received from pressure transducer 3026 of fluid delivery system 3020 and information (e.g., calibration information) stored in memory 3024.

With reference to instructions stored in memory, processor 3022 can also determine a FFR for tandem lesions. Using lesions 3006A and 3006B as an example, such a determination can be made according to the following equations:

$$FFR ( A ) pred = \frac{Pd - ( Pm / Pa ) Pw}{ Pa - Pm + Pd - Pw}$$

$$FFR ( B ) pred = \frac{( Pa - Pm ) ( Pm - Pd )}{pS ( pM - Pw )}$$

In the equations above, P_{d} is the distal pressure, Pₘ is the middle pressure, and Pₐ is the proximal pressure, which may also be referred to as the mean aortic pressure. In addition, P_{w} in the equation above is wedge pressure, which is the distal coronary pressure measured by pressure sensor 3018 of guidewire 3004 during balloon occlusion. The wedge pressure may be determined by processor 3022 and/or stored in memory 3024 based on a pressure measurement received from pressure sensor 3018 of guidewire 3004. The pressure measurement may be made during balloon occlusion (e.g., percutaneous transluminal coronary angioplasty) of stenotic lesion 3006A and/or lesion 3006B. Once determined, processor 3022 may store the characteristic information (e.g., calculated FFR values) in memory 3024, control a display communicatively coupled to the processor to display the FFR values or an indication thereof, and/or perform other suitable tasks.

Various examples have been described. These and other examples are within the scope of the following claims.

1. A method comprising:
positioning a sensor carried by a guidewire distally of a location of interest in a patient;
advancing a sensor delivery device over the guidewire and positioning a sensor of the sensor delivery device proximal to the location of interest in the patient; and
comparing a signal generated by the sensor carried by the guidewire to a signal generated by the sensor of the sensor delivery device and determining therefrom a characteristic of the location of interest.

2. The method of claim 1, wherein the location of interest comprises a lesion in a blood vessel of the patient.

3. The method of claim 1, wherein the sensor carried by the guidewire comprises an integrated sensor at a distal end of the guidewire.

4. The method of claim 1, wherein the sensor delivery device comprises a distal sleeve and a proximal portion and the distal sleeve has a guidewire lumen for sliding over and receiving the guidewire.

5. The method of claim 1, wherein the sensor of the sensor delivery device is located on one of the distal sleeve and the proximal portion.

6. The method of claim 5, wherein the proximal portion of the sensor delivery device comprises having a main section extending proximally from the distal sleeve and a distal transition extending distally from the main section, wherein the distal transition is fixedly coupled to an outer surface of the distal sleeve, the proximal portion comprises a communication channel for communicating a signal from the sensor of the delivery device to a location outside of the patient, and the proximal portion is adapted to facilitate positioning of the sensor of the delivery device within an anatomical structure of the patient.

7. The method of claim 1, wherein the characteristic of the location of interest comprises fractional flow reserve (FFR).

8. The method of claim 1, wherein comparing the signal generated by the sensor carried by the guidewire to the signal generated by the sensor of the sensor delivery device comprises determining a ratio of the signal generated by the sensor carried by the guidewire to the signal generated by the sensor of the sensor.

9. The method of claim 1, wherein the sensor carried by the guidewire comprises a fluid pressure sensor and the sensor of the sensor delivery device comprises a fluid pressure sensor.

10. The method of claim 1, wherein the location of interest comprises a first location of interest, and further comprising a second location of interest located proximally to the first location of interest.

11. The method of claim 10, wherein positioning the sensor of the sensor delivery device proximal to the location of interest comprises positioning the sensor of the sensor delivery device between the first location of interest and the second location of interest.

12. The method of claim 11, further comprising generating a signal representative of blood pressure at a location proximal to the second location of interest with an additional sensor.

13. The method of claim 12, wherein the additional sensor is a hemodynamic pressure transducer of a fluid injection system located outside of a body of the patient.

14. The method of claim 13, further comprising positioning a fluid tubing adapted to provide fluid communication between the fluid injection system and the patient proximally of the second location of interest.

15. The method of claim 12, wherein comparing the signal generated by the sensor carried by the guidewire to the signal generated by the sensor of the sensor delivery device comprises comparing the signal generated by the sensor carried by the guidewire to the signal generated by the sensor of the sensor delivery device and the signal representative of blood pressure at the location proximal to the second location of interest and determining therefrom a characteristic of the first location of interest and a characteristic of the second location of interest.

16. The method of claim 15, wherein the characteristic of the first location of interest and the characteristic of the second location of interest each comprise fractional flow reserve (FFR).

17. The method of claim 10, wherein the first location of interest comprises a first lesion in a blood vessel of the patient and the second location of interest comprises a second lesion in the blood vessel of the patient.

18-34. (canceled)

35. A method comprising:
positioning a sensor contained within a guidewire distally of a lesion in a blood vessel of a patient, the sensor contained within the guidewire being configured to generate a first signal representative of fluid pressure;
advancing a sensor delivery device having a sensor, a distal sleeve, and a proximal portion distally over the guidewire and positioning the sensor of the sensor delivery device proximal to the lesion, the sensor of the sensor delivery device being configured to generate a second signal representative of fluid pressure; and
providing an assessment of the location of interest based on a ratio of the first signal to the second signal.

36. The method of claim 35, wherein positioning the sensor carried by the guidewire comprises inserting the guidewire into the patient in a leading direction extending proximally to distally.

37. The method of claim 35, wherein the sensor carried by the guidewire comprises an integrated sensor at a distal end of the guidewire.

38. The method of claim 35, wherein the sensor of the sensor delivery device is located on one of the distal sleeve and the proximal portion.

39. The method of claim 38, wherein the proximal portion of the sensor delivery device comprises having a main section extending proximally from the distal sleeve and a distal transition extending distally from the main section, wherein the distal transition is fixedly coupled to an outer surface of the distal sleeve, the proximal portion comprises a communication channel for communicating a signal from the sensor of the delivery device to a location outside of the patient, and the proximal portion is adapted to facilitate positioning of the sensor of the delivery device within an anatomical structure of the patient.

40. The method of claim 35, wherein the characteristic of the location of interest comprises fractional flow reserve (FFR).

41. The method of claim 35, wherein the sensor carried by the guidewire comprises a fluid pressure sensor and the sensor of the sensor delivery device comprises a fluid pressure sensor.

42. The method of claim 35, wherein the location of interest comprises a first location of interest, and further comprising a second location of interest located proximally to the first location of interest.

43. The method of claim 42, wherein positioning the sensor of the sensor delivery device proximal to the location of interest comprises positioning the sensor of the sensor delivery device between the first location of interest and the second location of interest.

44. The method of claim 43, further comprising generating a signal representative of blood pressure at a location proximal to the second location of interest with an additional sensor.

45. The method of claim 44, wherein the additional sensor is a hemodynamic pressure transducer of a fluid injection system located outside of a body of the patient.

46. The method of claim 45, further comprising positioning a fluid tubing adapted to provide fluid communication between the fluid injection system and the patient proximally of the second location of interest.

47. The method of claim 42, wherein the second location of interest comprises a second lesion in the blood vessel of the patient.

48-57. (canceled)
